(19) United States
(12) Patent Application Publication
(10) Pub. No.: US 2026/0201367 A1
(43) Pub. Date: Jul. 16, 2026

(54) HEADPIECES FOR DNA-ENCODED CHEMICAL LIBRARIES

(71) Applicant: UNIVERSITE DE MONTREAL, Montreal (CA)

(72) Inventors: Marc-Andre POUPART, LAVAL (CA); Julien POUPART, LAVAL (CA); Christian LE GOUILL, MONTRÉAL (CA); Antoine DOUCHEZ

(21) Appl. No.: 19/135,330

(22) PCT Filed: Dec. 22, 2023
(86) PCT No.: PCT/CA2023/051751

§ 371 Date: Jun. 3, 2025

Related U.S. Application Data
(60) Provisional application No. 63/476,720, filed on Dec. 22, 2022.

(51) Int. Cl. C12N 15/10; C12Q 1/25; C40B 40/06

(52) U.S. Cl. CPC C12N 15/1068; C12Q 1/25; C40B 40/06; G01N 2333/9015

(57) ABSTRACT

The present document relates to headpieces for DNA-encoded chemical libraries and processes for preparing them, to DNA-encoded chemical libraries and DNA-supported pharmacophores or control molecules comprising the headpiece, and to uses thereof. The present document further relates to a method for generating multidimensional selection maps for a DNA-encoded library (DEL) (e.g., double-stranded or single-stranded DEL) against a single target.

### TECHNICAL FIELD

This technology generally relates to headpieces for DNA-encoded chemical libraries and processes for preparing them, to DNA-encoded chemical libraries and DNA-supported pharmacophores or control molecules comprising the headpiece, and to uses thereof.

### BACKGROUND

In the last decade, the use of DNA-encoded chemical libraries (DELs) for early hit identification has gained in importance in the field of medicinal chemistry. Due to their unparalleled size, with some libraries containing billions of different compounds, DELs offer unique opportunities to life scientists for the identification of novel chemical entities with specific binding profile. DNA-encoded libraries often cover very a large chemical space, and a single library can be reused many times for biological screens, making the process extremely efficient. Most often, biological targets are screened using a simple pulldown-type assay. Briefly, the purified protein of interest (POI) is immobilized on solid support, incubated with the library, washed with buffers until the desired level of enrichment is attained, followed by denaturation of the protein-binder complexes, elution of the binders and identification by PCR amplification followed by sequencing (e.g., next-generation sequencing (NGS)).

DELs can be divided into two distinct groups, depending on whether they rely on single- or double-strand encoding. While single-stranded DNA (ssDNA) has been used successfully in the past, the technology is inherently limited by the exposed nature of the nucleic bases which reduce the chemical tolerance of the oligonucleotide to various chemical transformations. Moreover, ligation of the ssDNA tags is far more tedious since enzymatic ligation cannot be done directly. Alternatives to circumvent this issue includes the use of splint ligation, which is operationally more complex and potentially costly, and the use of Huisgen copper catalyzed cycloaddition between oligonucleotides strands bearing both alkyne and azido functionalities, a chemical transformation which is known to induce significant damages to the coding DNA, and therefore this technology is limited to 3 synthetic cycles (or less) and the resulting libraries may be more challenging to amplify.

The use of double-stranded DNA (dsDNA) encoding is therefore far more common in commercial libraries, as it allows for maximum chemical tolerance of the DNA moiety, and is compatible with “sticky-end” enzymatic ligation of the tags. However, in order for dsDNA to be used, a headpiece (HP) must connect the pharmacophore to both DNA strands, as to avoid strand separation during reactions and HPLC purifications. In some cases, ssDEL may be preferred to dsDEL since dsDEL may be more limited in terms of screening modalities. Therefore, a technology that could combine the chemical resistance and ease of tag introduction of dsDEL with the versatility of ssDEL would be highly desirable.

Different headpieces have been previously described and are commercially available, but have significant drawbacks including reduced chemical stability, lower DNA ligation yields, rely on reversible conjugation chemistry, and/or lack of versatility. There thus remains a need for the development of alternative headpieces for use in DNA-encoded chemical libraries (DELs).

### SUMMARY

According to one aspect, the present technology relates to headpieces for DNA-encoded chemical libraries and processes for preparing them, to DNA-encoded chemical libraries and DNA-supported pharmacophores or control molecules comprising the headpiece, and to uses thereof. More specifically, the following embodiments are provided:

Embodiment 1: A headpiece, wherein said headpiece comprises a first polynucleotide sequence and a second polynucleotide sequence, wherein:
said second polynucleotide sequence comprises a sequence that is complementary to the first polynucleotide sequence;
at least part of said first and second polynucleotide sequences forms a double stranded polynucleotide sequence;
said first and second polynucleotide sequences are covalently and irreversibly linked to each other through at least one modified base pair present in the double stranded polynucleotide sequence; and
said first and second polynucleotide sequences each comprise a first and a second end, wherein:
   one of the first ends comprises a functionalizable moiety attached through a linker;
   the other of the first ends comprises a chain termination nucleotide; and
   the second ends comprise a DNA anchor site.

Embodiment 2: A headpiece, wherein said headpiece comprises a first polynucleotide sequence (e.g., sense strand) and a second polynucleotide sequence (e.g., antisense strand), wherein:
said second polynucleotide sequence comprises a sequence that is complementary to the first polynucleotide sequence;
at least part of said first and second polynucleotide sequences forms a double stranded polynucleotide sequence;
said first and second polynucleotide sequences are covalently and irreversibly linked to each other through at least one modified base pair present in the double stranded polynucleotide sequence; and
said first and second polynucleotide sequences each comprise a 5′- and a 3′-end, wherein:
   the 5′ end of the first polynucleotide sequence comprises a functionalizable moiety attached through a linker;
   the 5′ end of the second polynucleotide sequence comprises a chain termination nucleoside, preferably a dideoxynucleoside;
   the second polynucleotide sequence comprises one or more deoxy-uridine residues used as enzymatic cleavage sites; and
   the 3′ end of the first polynucleotide sequence comprises a two-nucleotide single strand overhang and the 5′ ends of the second polynucleotide sequence comprise a 5′-phosphate, thereby creating a DNA anchor site.

Embodiment 3: A DNA-encoded library (DEL) (e.g., double-stranded or single-stranded DEL) comprising a plurality of DNA-supported pharmacophores as defined herein.

Embodiment 4: Use of the headpiece as defined herein, for the preparation of a DNA-encoded library (DEL) (e.g., double-stranded or single-stranded DEL).

Embodiment 5: Use of the headpiece as defined herein, for anchoring a control molecule, preferably for use in a pulldown biological assay.

Embodiment 6: A method for the preparation of a headpiece as defined herein, the method comprising:
preparing a 6-vinyl purine precursor;
preparing the first polynucleotide sequence comprising the 6-vinyl purine precursor and converting the 6-vinyl purine precursor into a 6-vinyl purine (e.g., sense strand);
preparing the second polynucleotide sequence (e.g., antisense strand); and
contacting the first polynucleotide sequence and second polynucleotide sequence to form a covalently linked double stranded (HP) wherein the vinyl group of the 6-vinyl purine reacts with an amine group of a base facing the 6-vinyl purine on the second sequence.

Embodiment 7: A method for preparing a DNA-encoded library (DEL) (e.g., double-stranded or single-stranded DEL), said method comprising:
preparing the headpiece as defined herein, optionally wherein the headpiece comprises an integrated primer;
contacting the headpiece with a pharmacophore comprising a functional group reactive to the functionalizable moiety to covalently link the pharmacophore with the functionalizable moiety;
optionally, attaching a primer to the DNA anchor site of the headpiece; and
optionally, attaching at least one DNA tag sequence to the primer.

Embodiment 8: A method for adding secondary molecular effectors to a DNA-encoded library (DEL) (e.g., double-stranded or single-stranded DEL), said method comprising:
a) leaving the second polynucleotide sequence of the headpiece as defined herein at the enzymatic cleavage sites by treatment with a uracil-specific excision reagent (e.g. USER™ 1, II or III, preferably USER™ I or III);
b) introducing a first molecular effector by hybridizing a first functionalized single-stranded oligonucleotide of complementary sequence 5′ of the crosslinking base-pair, and ligating with T-4 DNA ligase;
c) repeating step (b) as required until the length of the functionalized second polynucleotide sequence remains shorter that that of the first polynucleotide sequence of the headpiece as defined herein; and
d) optionally, adding one last, non-covalently bound, secondary molecular effector by hybridizing a functionalized single-stranded oligonucleotide of complementary sequence 3′ of the crosslinking base-pair.

Additional objects and features of the technology will become more apparent upon reading of the following non-restrictive description of exemplary embodiments and examples section, which should not be interpreted as limiting the scope of the invention.

### DESCRIPTION OF THE FIGURES

FIG. 1 shows the structure of a headpiece (HP-1), according to one example embodiment. AOP=Amino-PEG₄-propionate.

FIG. 2A shows the sequences of oligonucleotide precursors antisense HP strand (AS_HP-1; SEQ ID NO: 1), sense HP strand (S_HP-1; SEQ ID NO: 2), and of crosslinked HP-1. FIG. 2B shows the structure of precursor X (6-vinyl purine) within the sequence as cytidine crosslinker under acidic conditions, and precursor Y (5′-(amino-PEG3)). FIG. 2C shows the structure of precursors X′, and two variant of precursors Y′ (Fmoc-Y′ and MTr-Y′) before insertion into or on the sequence to form precursors X and Y, respectively.

FIG. 3A shows the sequence of HP-1 with Primer 1. FIG. 3B shows the synthesis of HP-1 from oligonucleotides precursors AS_HP-1 and S_HP-1, as well as enzymatic ligation of Primer 1.

FIG. 4A shows the structure and sequence of HP-2 (from oligonucleotides precursors AS_HP-2 [SEQ ID NO: 5] and S_HP-2 [SEQ ID NO: 6]) with integrated Primer 1 (dashed underlined). FIG. 4B shows the structure and sequence of HP-3 (from oligonucleotides precursors AS_HP-3 [SEQ ID NO: 7] and S_HP-3 [SEQ ID NO: 8]) with integrated Primer 1 (underlined), a reversible affinity-pairing region (boxed), and two enzymatically-cleavable residues.

FIG. 5A shows the results of a gel electrophoresis for HP-1 on a non-denaturing agarose gel and FIG. 5B shows the results of a denaturing polyacrylamide gel (15% TBE-Urea), with starting material as controls.

FIG. 6 shows a schematic and results of the comparative enzymatic ligation efficiency between HP-A and HP-1.

FIG. 7 shows the synthesis of full-length DNA-supported vemurafenib derivatives and the result of a 2-round selection against solid-supporter BRAF (Vmb1-AOP-HP-1-P1-T1-T2-T3-T4-CP indicated by arrow).

FIG. 8 shows TR-FRET dose-response curves for HP-2 and HP-A-supported vemurafenib derivatives against His-tagged BRAF kinase domain.

FIG. 9 shows the application of ssDEL to the screening of protein targets in solution.

FIG. 10A shows a schematic representation of enzyme-mediated conversion of HP-2 to partially single strand HP-2 (p-ssHP-2), followed by the enzymatic ligation of biotinylated DNA. FIG. 10B and FIG. 10C show detailed sequences and reaction analysis by denaturing polyacrylamide gel electrophoresis (FIG. 10B: strand release; FIG. 10C: Biotin addition).

FIGS. 11A-11B show a schematic representation of an application of HP-2 in the context of covalent DEL performed with the target in solution (FIG. 11A) and with a solid supported target (FIG. 11B).

FIGS. 12A-12C show advanced screening modalities enabled by ss/dsHP-3: in-solution POI screening with reversible protein crosslinking (FIG. 12A); in cellulo- (FIG. 12B) and membrane-bound (FIG. 12B) screens; cooperative screen to molecular glues (FIG. 12D).

FIG. 13 shows the concept of Dynamically directed libraries for in cellulo screening against multiple proteins in parallel.

FIG. 14 Illustrate the advantage of combining screening modalities for the screening of covalent DEL; Hypothetical selection map of a single hit in two settings.

FIG. 15 shows a schematic representation of the designs of the headpieces HP-A, HP-B, and HP-1/HP-2.

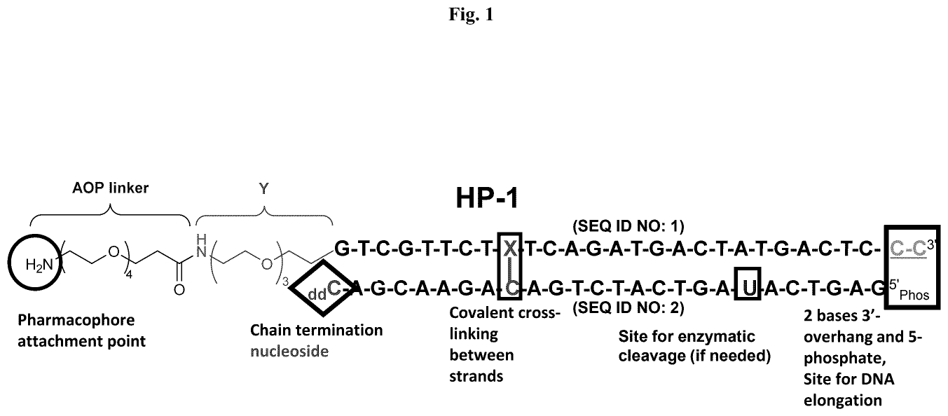

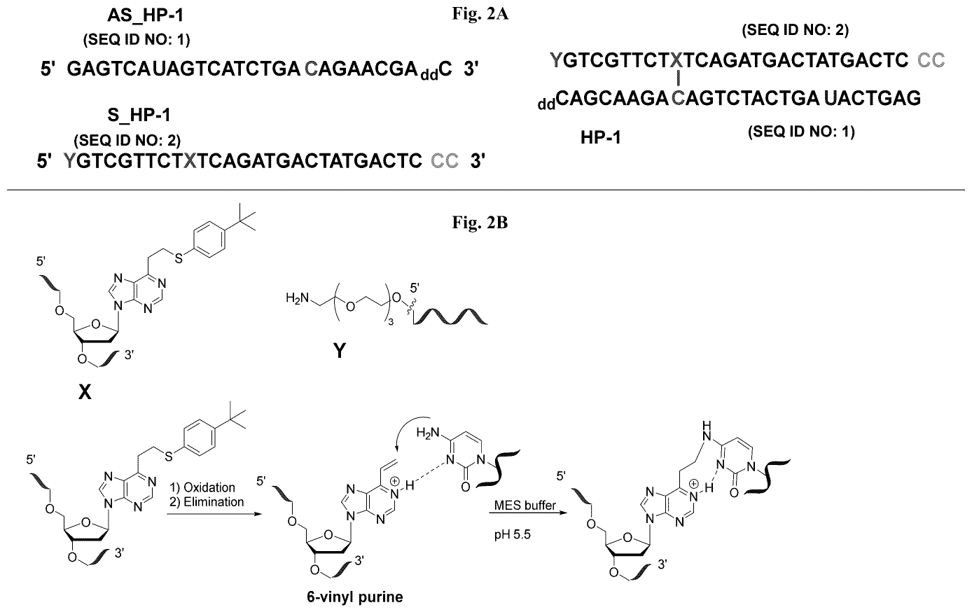

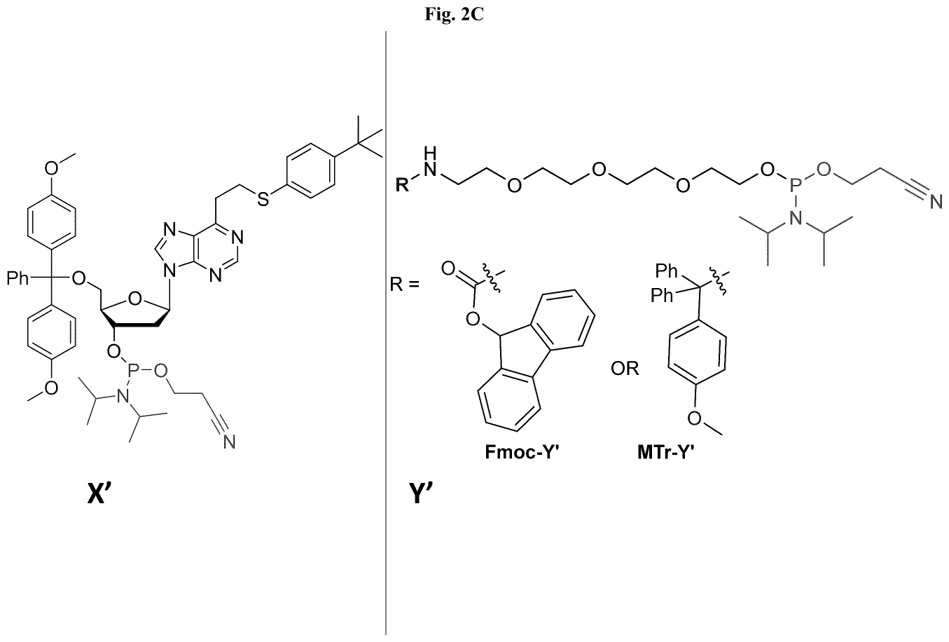

### SEQUENCE LISTING

This application contains a Sequence Listing in computer readable form created on Dec. 20, 2023. The computer readable form is incorporated herein by reference.

TABLE 1
Sequence Listing

| SEQ ID NO: | Description |
|---|---|
| 1 | Antisense HP-1 strand (AS_HP-1 (5′-3′)) |
| 2 | Sense HP-1 strand (S_HP-1 (5′-3′)) |
| 3 | Primer 1 forward strand (5′-3′) |
| 4 | Primer 1 reverse strand (5′-3′) |
| 5 | Antisense HP-2 strand (AS_HP-2 (5′-3′)) |
| 6 | Sense HP-2 strand (S_HP-2 (5′-3′)) |
| 7 | Antisense HP-3 strand (AS_HP-3 (5′-3′)) |
| 8 | Sense HP-3 strand (S_HP-3 (5′-3′)) |
| 9 | HP-2/3 PCR Probe forward strand (5′-3′) |
| 10 | Biotinylated ssDNA |

### DETAILED DESCRIPTION

All technical and scientific terms and expressions used herein have the same definitions as those commonly understood by a person skilled in the art to which the present technology pertains. The definition of some terms and expressions used is nevertheless provided below. To the extent the definitions of terms in the publications, patents, and patent applications incorporated herein by reference are contrary to the definitions set forth in this specification, the definitions in this specification will control. The section headings used herein are for organizational purposes only and are not to be construed as limiting the subject matter disclosed.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition or mixture containing a component also contemplates a mixture of two or more components. It should also be noted that the term “or” is generally employed in its sense including “and/or” unless the context clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, as per the practice in the art. Alternatively, “about” can mean a range of up to 20%, preferably up to 10%, more preferably up to 5%, and more preferably still up to 1% of a given value. Where particular values are described in the application and claims, unless otherwise stated the term “about” meaning within an acceptable error range for the particular value should be assumed.

The term “pharmacophore” as used herein refers to an organic molecule or part of an organic molecule having a potential biological activity. The term herein equally refers to test molecules attached to a headpiece in a DNA-encoded library or to a known molecule used as a positive or negative control.

The terms “DNA-encoded library”, “DNA-encoded chemical library” or “DEL” as used herein refer to pharmacophores attached to DNA fragments that serve as identification bar codes. The DNA fragments of the present DNA-encoded libraries comprises at least a headpiece as defined herein to which the pharmacophore is attached through a linker.

As used herein, the term “oligonucleotide” or “polynucleotide” refers to a polymeric form of nucleotides that may have various lengths (e.g., 5-500 bases), including but not limited to deoxyribonucleotides and/or ribonucleotides, or analogs or modifications thereof. The term includes both single-stranded (ss) and double-stranded (ds) molecules. Double-stranded molecules contain two strands that may be partially or completely complementary to each other. Oligonucleotide sequences that are “complementary” are those that are capable of base-pairing according to the standard Watson-Crick complementarity rules (e.g., A-T [or U] and G-C base pairing).

As used herein, the term “headpiece” refers to a molecule that connects the pharmacophore to either one or both of the DNA strands, commonly used in DNA-encoded libraries. In some aspects, the purpose of the headpiece is to avoid strand separation during reactions and HPLC purifications. Headpieces may include any number of oligonucleotides, linkers (e.g., PEGs [Amino-PEG₄-propionate], for instance sinker through a deoxy nucleotide, such as 5-amino-PEG3-deoxy-guanosine), spacers, functionalizable moieties, and/or hairpin structures.

The present technology generally related to a headpiece for use in the preparation of DNA-encoded libraries. Preferably, the present headpiece comprises a double stranded polynucleotide sequence which comprises, within its sequence, a covalent bond between its two strands, preferably an irreversible covalent bond.

For instance, the present headpiece comprises a first polynucleotide sequence (sense strand) and a second polynucleotide sequence (antisense strand), wherein:
said second polynucleotide sequence comprises a sequence that is complementary to the first polynucleotide sequence;
at least part of said first and second polynucleotide sequences forms a double stranded polynucleotide sequence;
said first and second polynucleotide sequences are covalently and irreversibly linked to each other through at least one modified base pair present in the double stranded polynucleotide sequence; and
said first and second polynucleotide sequences each comprise a first and a second end, wherein:
   one of the first ends comprises a functionalizable moiety attached through a linker;
   the other of the first ends comprises a chain termination nucleotide; and
   the second ends comprise a DNA anchor site.

In other embodiments, the present headpiece comprises a first polynucleotide sequence (sense strand) and a second polynucleotide sequence (antisense strand), wherein:
said second polynucleotide sequence comprises a sequence that is complementary to the first polynucleotide sequence;
at least part of said first and second polynucleotide sequences forms a double stranded polynucleotide sequence;
said first and second polynucleotide sequences are covalently and irreversibly linked to each other through at least one modified base pair present in the double stranded polynucleotide sequence; and
said first and second polynucleotide sequences each comprise a 5′- and a 3′-end, wherein:
   the 5′ end of the first polynucleotide sequence comprises a functionalizable moiety attached through a linker;
   the 5′ end of the second polynucleotide sequence comprises a chain termination nucleoside, preferably a dideoxynucleoside;
   the second polynucleotide sequence comprises one or more deoxy-uridine residues used as enzymatic cleavage sites; and
   the 3′ end of the first polynucleotide sequence comprises a two-nucleotide single strand overhang and the 5′ ends of the second polynucleotide sequence comprise a 5′-phosphate, thereby creating a DNA anchor site.

In some examples, the modified base pair comprises a crosslink selected from an alkyl, alkylamine, or alkoxy group comprising a 3-atom chain. For example, the modified base pair is of Formula I:

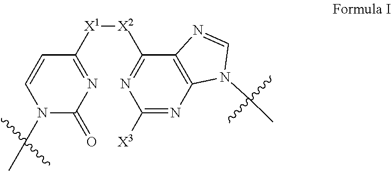

wherein:
X¹ and X² taken together form a —CR₂—CR₂—CR₂—, —CR₂—CR₂—NH—, —NH—CR₂—CR₂—, —CR₂—CR₂—O—, or —O—CR₂—CR₂— group, wherein R is independently H or methyl, preferably R is H in all instances, preferably X¹ and X² taken together form a —NH—CR₂—CR₂— group, most preferably a —NH—CH₂—CH₂— group;
X³ is selected from H and NH₂;
each of the base being attached to a phosphorylated sugar opposite to each other in the first and second polynucleotide sequences.

In some preferred examples, the modified base pair is located between the 4^{th} and 15^{th} base from the 5′-end of the sense oligonucleotide sequence, preferably between the 6^{th} and 10^{th} base, while the double stranded polynucleotide sequence may comprise from 15 to 35 base pairs, preferably from 20 to 30 base pairs.

Any of the first and second polynucleotide sequences may comprise the functionalizable moiety. In some examples, the functionalizable moiety is attached through a linker to the sense polynucleotide sequence. In those cases, the first end of the antisense polynucleotide sequence will comprise the chain termination nucleotide.

Examples of chain termination nucleoside include, without limitation, dideoxynucleosides, dimethoxynucleosides and 3-propynyloxy-deoxynucleosides, preferably a dideoxynucleoside, a dideoxycytidine.

In some preferred examples, deoxy-uridine residues, preferably one or two, are used as enzymatic cleavage point in the antisense strand, located between 1 and 6, preferably between 1 and 2 residues from the crosslinking base pair.

The linker attaching the functionalizable moiety to the polynucleotide sequence may comprise, for example, a polyether chain, preferably a di(ethylene glycol), tri(ethylene glycol), or tetra(ethylene glycol) chain, attached to the phosphorylated nucleotide present at the 5′ end of the polynucleotide sequence. For example, the functionalizable moiety is attached through a polyether chain attached to a deoxy-nucleotide, such as a PEG(2-5)-deoxy-nucleotide, e.g. PEG3-deoxy-guanosine linker.

The functionalizable moiety generally comprises a reactive group or atom that serves as an anchoring point for an organic molecule (e.g., pharmacophore to be tested in a DNA-encoded library). Such a reactive group or atom include an amine group, preferably a primary amine or azide group, or a functional group comprising a reactive moiety (e.g., an iodo-phenyl or bromo-phenyl group). For instance, the functionalizable moiety and its linker may be a 5′-amino-PEG-deoxy-nucleotide, with the PEG chain comprises between 2 and 5 ethylene glycol units, e.g. 5′-amino-PEG3-deoxy-guanosine.

The double stranded polynucleotide sequence of the headpiece may further comprise one or more cleavage sites. For example, the cleavage site may comprise a uridine in one of the first and second polynucleotide sequences, preferably in the antisense polynucleotide sequence. The cleavage sites may comprise one or more uridines (e.g., deoxy-uridines) in one of the first and second polynucleotide sequences, preferably in the second polynucleotide sequence.

In some examples, one of the first and second polynucleotide sequences comprises an antisense strand that is at least 70%, 80%, 90%, 95%, or 100% identical to the sequence of any one of SEQ ID NOs: 1, 5, and 7; and/or a sense strand that is at least 70%, 80%, 90%, 95%, or 100% identical to the sequence of any one of SEQ ID NOs: 2, 6, and 8.

Uses of the present headpiece are also contemplated, for instance, in the preparation of a DNA-supported pharmacophore, a DNA-encoded library, and/or for anchoring a control molecule, preferably for use in a pulldown biological assay.

Also described is a method for preparing a headpiece as defined herein, the process comprising the steps of:
a) preparing a 6-vinyl purine precursor;
b) preparing of the first sequence comprising the 6-vinyl purine precursor and converting the 6-vinyl purine precursor into a 6-vinyl purine (e.g. S-HP);
c) preparing of the second sequence (e.g. AS-HP); and
d) contacting the first sequence and second sequence to form a covalently linked double stranded (HP) wherein the vinyl group of the 6-vinyl purine reacts with an amine group of a base facing the 6-vinyl purine on the second sequence.

For instance, step (a) comprises preparing a compound of the Formula II:

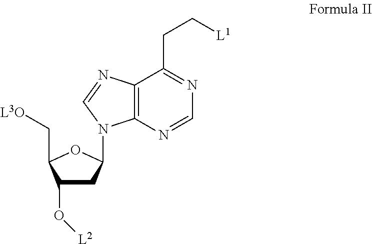

wherein L¹ is a leaving group, L² is an activated phosphorus moiety, and L³ is a protecting group. For example, L¹, L² and L³ are groups commonly known or usable in oligonucleotide synthesis, e.g. solid-supported oligonucleotide synthesis. Preferably, the compound of Formula II is of Formula II(a):

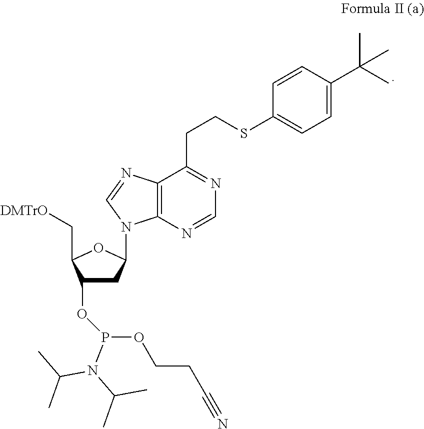

The present technology also relates to a DNA-supported pharmacophore comprising a headpiece as defined herein and a pharmacophore attached to the functionalizable moiety of the headpiece.

The DNA-supported pharmacophore may further comprise a primer attached to the DNA anchor site of the headpiece. In some cases, the primer may be attached to the headpiece. In some cases, the headpiece comprises an integrated primer (e.g., SEQ ID NOs: 5-8). Examples of primers comprise a forward strand that is at least 70%, 80%, 90%, 95%, or 100% identical to SEQ ID NO: 3, and/or a reverse strand that is at least 70%, 80%, 90%, 95%, or 100% identical to SEQ ID NO: 4.

Generally, the DNA-supported pharmacophore will further comprise at least one DNA tag sequence, for instance, between 2 and 5 DNA tag sequences, preferably 3 to 5 DNA tag sequences, more preferably 3-4 DNA tag sequences, wherein when said DNA tag sequences are sequentially ligated to the primer. Each of tag sequence may generally comprise between 5 and 12 base pairs, preferably between 6 and 10 base pairs. Preferably, a closing primer is also attached to the free end of the last DNA tag sequence.

In some aspects, the first and/or second polynucleotide sequences comprise nucleoside analogs, such as locked nucleic acid residues, 2′-alkylated RNA residues, peptide nucleic acid residues, and/or 2′-fluoro DNA residues.

In some aspects, the first and/or second polynucleotide sequences comprise phosphodiester or phosphonothioate linkages.

In some aspects, the said first and/or second polynucleotide sequences comprise an affinity-pairing region for binding to one or more secondary molecular effectors. In some aspects, said binding is reversible. In some aspects, the secondary molecular effectors comprise biotin groups, photoreactive groups, cell-penetrating peptides, secondary protein recruiters, and/or fluorophores.

A DNA-encoded library comprising a plurality of DNA-supported pharmacophores as defined herein is also contemplated as well as methods for preparing the same. For example, the method comprises the steps of:
a) preparing a headpiece as defined herein, optionally wherein the headpiece comprises an integrated primer;
b) contacting the headpiece of step (a) with a pharmacophore comprising a functional group reactive to the functionalizable moiety to anchor the pharmacophore to the functionalizable moiety;
c) optionally, attaching a primer to the DNA anchor site of the headpiece; and
d) attaching at least one DNA tag sequence to the primer.

For instance, the primer and DNA tag sequence(s) are as defined herein, and may further comprise a closing primer to the free end of the last DNA tag sequence.

Examples of reactions that may be carried for anchoring a pharmacophore to the functionalizable moiety of the headpiece in step (b) include, without limitation, an amide coupling, Suzuki coupling, Heck coupling, Buchwald-Hartwig coupling, Ullman coupling, Sonogashira coupling, Urea formation, etc. The functionalizable moiety of the headpiece may include an amine group (e.g. NH₂) or a functional group adapted for said coupling reaction, for example, attached to the amine group of the headpiece. For example, such functional group may include a p-iodobenzoyl or p-bromobenzoyl group.

The present technology also relates to a method for generating multidimensional selection maps for DNA-encoded library (DEL) (e.g., double-stranded or single-stranded DEL) members against a single target, said method comprising:
performing pulldown assays in at least two different modalities;
performing genomic analysis of each individual modality;
computing the difference in deduplicated, normalized counts for each member of the library across screening modalities; and
optionally, plotting the results to allow visual analysis.

In some aspects, if multiple rounds of selection were used for a given screening modality (e.g., in case of solid-supported targets), the active fraction (β) and retention (α) are derived using the following equations:

$$i . β = \frac{{( {count}_{( Round 1 )} )}^{2}}{{count}_{( R ound 2 )} * {count}_{( Initial )}} ; and$$

$$ii . α = \frac{{count}_{( Round 1 )}}{β * {count}_{( Initial )}} ;$$

wherein count₍ᵢₙᵢₜᵢₐₗ₎ represents the count number of each library member before the selection and count_{(Round 1)}/count_{(Round 2)} represents the same hits normalized count number after Round 1 or Round 2, optionally wherein the active fraction derived from the solid-supported multi-round selection is used with equation (ii) to calculate retention for all screening modalities.

In some embodiments, “multidimensional selection maps” refer to the variations of normalized count number associated with a single library member across screening modalities. Optionally, retention across screening modalities can also be added to the selection map.

In some embodiments, the pulldown assays in at least two different modalities refers to solid-supported assays, in-solution assays using modified protein, in-solution assays using unmodified protein, cell membrane-based assays, and/or in cellulo assays.

In some embodiments, performing genomic analysis of each individual modality comprises amplification followed by sequencing of the corresponding sequence using known methods.

In some embodiments, the DEL library is the DEL library defined herein. In some embodiments, the DNA-encoded library (DEL) (e.g., double-stranded or single-stranded DEL) comprises a plurality of DNA-supported pharmacophores as defined herein, comprising a headpiece as defined herein, and a pharmacophore attached to the functionalizable moiety of the headpiece.

The recitation of an embodiment or example for a variable herein includes that embodiment or example as a single embodiment or example or in combination with any other embodiments, examples or portions thereof. The recitation of an embodiment herein includes that embodiment as any single embodiment or in combination with any other embodiments or portions thereof.

### EXAMPLES

The following non-limiting examples are illustrative embodiments and should not be construed as further limiting the scope of the present invention. These examples will be better understood with reference to the accompanying figures.

Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained.

Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### Example 1—Materials and Methods

### Chemistry

Unless otherwise noted, all reactions were performed under nitrogen atmosphere. Glassware was stored in the oven (140° C.) or flame-dried and let cool down under vacuum and flushed with nitrogen before use. Dry solvents were either purchased commercially (pyridine, DMF) or distilled from CaH₂ before use (DCM) and stored under nitrogen atmosphere. DMT-MM was synthesized in-house according to published literature and fresh solutions (in Dnase-free water) were prepared for each use. All other starting material and reagents were purchased from commercial sources and used without further purification. Flash chromatography was performed using either 230-400 mesh silica gel from SiliCycle™ Inc. or on a Biotage Isolera™ One using prepacked FlashPure™ columns from Buchi. Large scale reactions were performed in 10 L tri-col flasks, were agitated mechanically and worked-up directly in the reaction vessel. Nuclear magnetic resonance spectra were recorded on a Bruker 400 MHz (¹H and ¹³C) or on a Bruker 600 MHz (³¹P). DNA incubation was performed in a Grant-bio PCH-1 dry block heater/cooler. DNA centrifugation was done either using a Fisherbrand microcentrifuge kept in a refrigerator (4° C.) (for 2.0, 1.5, 0.5 mL Eppendorf tubes and 0.2 mL PCR tubes) or a Thermoscientific Sorval™ ST Plus refrigerated centrifuge kept at 4° C. and set at 5000 rpm (15 and 50 mL Falcon™ tubes) or 4000 rpm (96-well plates). Analytical IP-HPLC were performed on an Agilent instrument using an Agilent AdvanceBio™ C18 column (2.7 microns, 2.1×50 mm) and solvents A: 10 mM TEAA buffer, pH 7.5 and B: 50% MeCN/svt A, with either gradient 1 (15-25% B/A), gradient 2 (10-25% B/A), gradient 3 (5-25% B/A), gradient 4 (5-45% B/A) or gradient 5 (15-25% B/A). Analytical HILIC-HPLC were performed on a Thermofisher HPLC using a Waters BEH Amide column (2.5 microns, 2.1×100 mm) and solvents A: 75:25 H₂O/MeCN, 8 mM NH₄OAc, 5 μM Medronic acid and B: 25:75 H₂O/MeCN, 8 mM NH₄OAc, 5 μM Medronic acid with a gradient of 45-90% A/B. In all cases, UV detection was done at 260 nm. Mass spectrometry analyses were done either at the Centre regional d'Analyse par spectrommetrie de masse (Montreal), or using a Waters BEH Amide column (2.5 microns, 2.1×100 mm) and solvents A: 75:25 H₂O/MeCN, 8 mM NH₄OAc, 5 μM Medronic acid and B: 25:75 H₂O/MeCN, 8 mM NH₄OAc, 5 μM Medronic acid with a gradient of 45-90% A/B coupled to a ThermoFisher™ Q-Exactive mass spectrometer, denoted MS(TOF) and MS(Orbitrap) respectively, and reported as the deconvoluted mass in Daltons (Da). Preparative IP-HPLC were performed on an Agilent preparative system using either a Waters XbridgeOligonucleotides™ BEH C18 column (2.5 microns, 10×50 mm) or a Waters XbridgeOligonucleotides™ BEH C18 column (2.5 microns, 19×50 mm) and solvents A: 100 mM TEAA buffer, pH 7.5 and B: 95% MeCN/water, with either gradient 1(5-25% B/A) or gradient 2 (5-45% B/A), with UV monitoring at 260 nm. Optical density measurements were recorded on a ThermoScientific NanoDrop™ One, with the following conversion factor (omitting the mass contribution of the pharmacophore):

$$\frac{1. OD}{( Dilution factor )} = 50 ( μg / mL ) dsDNA = {[ C ]}_{( μ g / mL )}$$

And:

$${[ C ]}_{( mM )} = \frac{{[ C ]}_{( μ g / mL )}}{{MW}_{DNA}}$$

$${n}_{( nmol )} = {[ C ]}_{( mM )} \times {( total solution volume )}_{( μ L )}$$

### Oligonucleotide Synthesis

Oligonucleotides S_HP-1 and AS_HP-1 were synthetized externally by Integrated DNA Technologies (IDT). S_HP-2 and AS_HP-2 were synthetized externally by either Boc Sciences Inc., Polaris Oligonucleotides Inc., or Galenvs Sciences Inc. Modified oligonucleosides X and Y were supplied as the active phosphoramidite X′ and Y′ (see below) and shipped in silanized glass vials, under nitrogen atmosphere and in dry ice. S_HP-1 or S-HP-2 was purified by HPLC while AS_HP-1 or AS-HP-2 was purified by standard desalting, and both oligonucleotides were supplied as ~1 mM solution in water, with sodium as DNA counter-ion. Both oligonucleotide strands were analyzed for quality (LCMS, gel electrophoresis) and exact concentration (optical density).

### DNA-Supported Chemistry

All buffers were made in-house from commercial reagents, using DNase-free water purchased form VWR and are reported as follow: Buffer (Volume, [Concentration], pH). Reactions were performed in either 0.2-, 0.5-, 1.5- or 2.0-mL DNase-free microcentrifuge tubes or 15- or 50-mL Falcon™ tubes. DNA precipitation and sodium exchange were done by adding 0.1 volumes of either 3 M NaOAc (for oligos over 40 bp) or 3 M NaOAc containing 0.3 M MgCl₂ (for oligos under 40 bp) followed by 4.0 volumes of absolute ethanol. The vials were then left at −20° C. for a minimum of 2 h, centrifuged at 5 000 rpm in a refrigerated centrifuge (4° C.) for 45 minutes and decanted. The sides of the vial were wiped with a Kimwipe™. The mother liquor was kept in a separate vial until the end of the experiment. The precipitated DNA is then washed with 2 volumes of cold (−20° C.) absolute ethanol, decanted, and dried under high vacuum for 5 minutes to give a white solid. The precipitated DNA is dissolved in water of buffer at ~1 mM concentration. In cases where the solution obtained is cloudy, a second centrifugation can be done to pellet the insoluble material, and the solution is transferred to a fresh microcentrifuge tube. Optical density needs to be assessed after precipitation to insure acceptable (>80%) DNA recovery and determine exact concentration. Agarose gel electrophoresis was performed on a FisherBrand horizontal electrophoresis module and FisherBrand™ power supply using 33% Sybr™Red-stained agarose and eluted at 110 mV for 80 minutes before visualization. Polyacrylamide gel electrophoresis (PAGE) was performed using an Invitrogen Xcell SureLock™ vertical system, a FisherBrand power supply, and Invitrogen 15% TBE-Urea precast gels (used within 4 weeks of reception). Samples were diluted in loading buffer and heated at 70° C. for 3 minutes to denature strands, put directly on ice and loaded on the gel. Elution was done at 180 mV for 90 minutes, followed by a post migration staining using SybrGreen II stain (10 000× dilution). The gel was then visualized directly. Both polyacrylamide and agarose gels were referenced using Ultra-low range DNA-ladder (10-300 bp dsDNA) from ThermoScientific. USER™ (combination of Uracil DNA-glycosylase and DNA glycosylase-lyase Endonuclease VIII), T-4 ligase, KLENOW™, USER™ and A-exonuclease enzymes were purchased from New England Biolabs and kept at −20° C. until use. Enzyme dilutions were made using New England Biolabs Diluent A (10 mM Tris-HCl pH=7.4, 0.1 mM EDTA, 1 mM DTT, 200 μg/mL BSA, 50 mM KCl, 50% glycerol).

### Ligations and Ligation Efficiency Determination

In a 1.5 mL microcentrifuge tube, AOP-HP-A or AOP-HP-1 (20.0 μL, 1.00 mM in DNase-free water, 20 nmol) were diluted with 10× Ligation Buffer (4.6 μL) and treated successively with Primer 1 (7.8 μL, 1.53 mM in DNase-free water, 12.0 nmol) and T-4 enzyme (50 u/uL in water) (0.9 μL, 50 U/μL, 45 U). The vial was thoroughly vortexed and left to stand at room temperature for ca 18 h, after which reaction were precipitated and analyzed by IP-LCMS. Conversion was calculated by integrating signals associated with starting material and desired product. Since reaction on AOP-HP-A (the HP described in WO2005058479A2) only showed 52% conversion, the ligation step was repeated using the same amounts and reaction time, yielding to full conversion.

Both headpieces were resuspended TEAA buffer (20.0 μL, 10 mM, pH=7.6) and transferred to a 96 deep-well plate. The crude products were each purified by preparative IP-HPLC, and relevant fractions were frozen in dry ice and lyophilized. The content of the fractions was then dissolved in water containing 20% of MeCN, combined and transferred to a single 1.5 mL microcentrifuge tube. The content of the tube was then lyophilized, followed by two more lyophilization from water containing 20% of MeCN to give HP-1 as white fluffy solid which was dissolved in DNase-free water (20.0 μL) and analyzed by IP-HPLC (gradient 1).

### Example 2—Preparation of Nucleoside Precursor X′

The nucleoside Precursor X′ was prepared according to the procedure illustrated in Scheme 1.

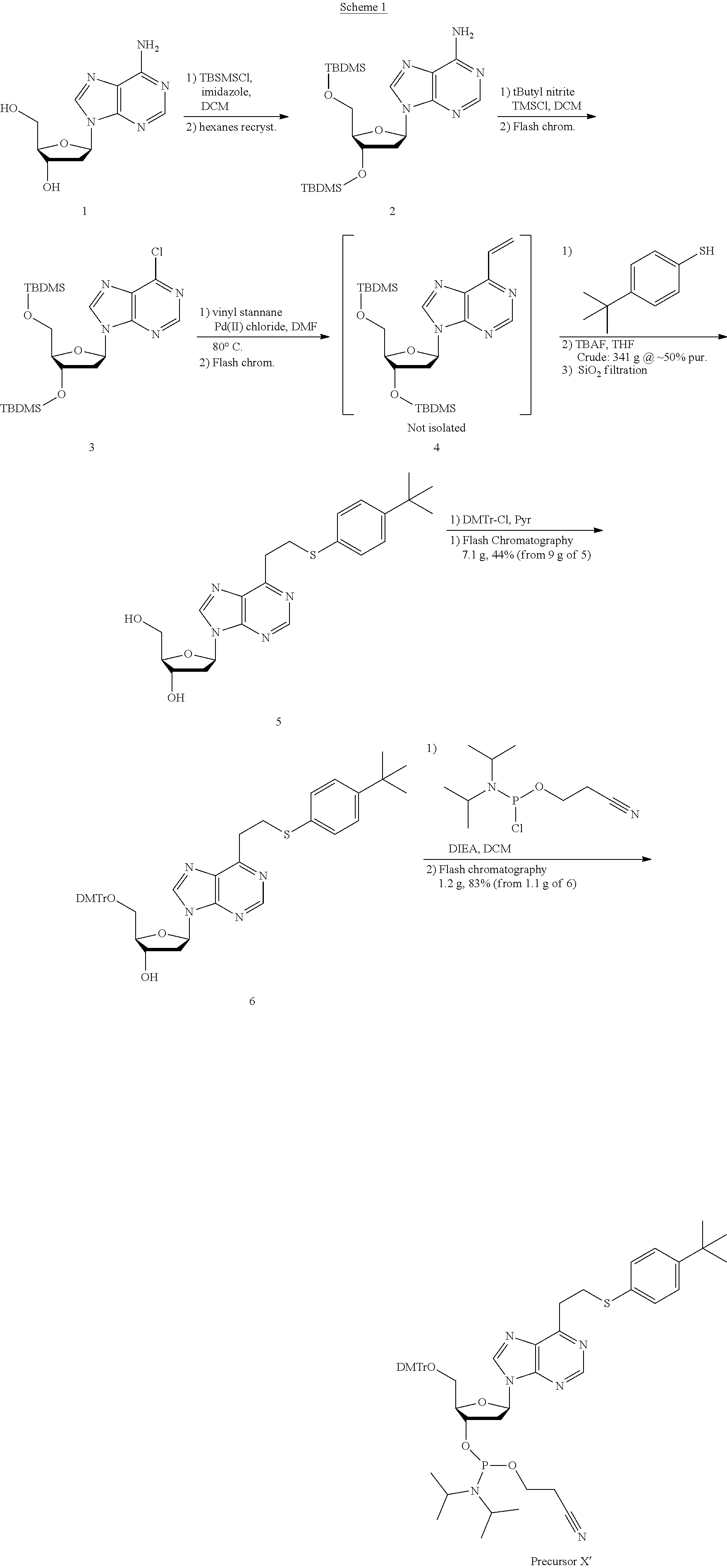

### (i) Preparation of Intermediate 2 (9-((2R,4S,5R)-4-((tert-butyldimethylsilyl)oxy)-5-(((tert-butyldimethylsilyl)oxy)methyl)tetrahydrofuran-2-yl)-9H-purin-6-amine)

To a cold (~0° C.) mixture 2′-deoxy-adenosine hydrate (100 g, 371 mmol) and imidazole (167 g, 2.45 mol) in dry DMF (800 mL) was added solid tert-butylchlorodimethylsilane (179 g, 1.19 mmol). The clear solution was stirred overnight at room temperature. The following morning, MeOH was added (100 mL) and the mixture was concentrated in vacuo. The residue was dissolved in DCM (400 mL), treated with saturated NaHCO₃ (600 mL) and the mixture was stirred vigorously for 5 minutes. The organic layer was decanted, and the aqueous layer was extracted with DCM (3×250 mL). The combined organic layers were washed with water (500 mL) and brine (500 ml), dried over sodium sulfate, and filtered. Solvent was evaporated and the residue was co-evaporated twice with toluene (300 mL). The obtained solid was transferred to a Buchner filter and slowly triturated with hexanes (3×100 mL), followed by evaporation of remaining volatiles under high vacuum gave bis-protected deoxyadenosine Intermediate 2 as white solid (182.34 g, quantitative yield). The NMR data below correspond to previously reported results.

¹H-NMR (400 MHz, CDCl₃) δ 0.10-0.11 (m, 12H), 0.92 (s, 18H), 1.84 (br. S, 1H), 2.41-2.47 (m, 1H), 2.61-2.68 (m, 1H), 3.76-3.80 (m, 1H), 3.86-3.90 (m, 1H), 4.01-4.03 (m, 1H), 4.61-4.64 (m, 1H), 5.63 (br. S, 2H), 6.46 (t, J=4 Hz, 1H), 8.14 (s, 1H), 8.36 (s, 1H); ¹³C-NMR (100 MHz, CDCl₃) δ −5.5, −4.8, −4.7, 18.0, 18.4, 25.8, 26.0, 41.3, 62.8, 71.9, 84.3, 87.9, 120.1, 139.1, 149.6, 152.9, 155.3. MS calculated for C₂₂H₄₂N₅O₃Si₂ [M+H]⁺ 480.3, found 480.5.

### (ii) Preparation of Intermediate 3 (9-((2R,4S,5R)-4-((tert-butyldimethylsilyl)oxy)-5-(((tert-butyldimethylsilyl)oxy)methyl)tetrahydrofuran-2-yl)-6-chloro-9H-purine)

To a cold (0° C.) solution of Intermediate 2 (135 g, 281 mmol) in DCM (1.38 L) was added trimethylsilyl chloride (72 mL, 563 mmol) dropwise over 5 minutes, followed by the dropwise addition of a solution of tert-butylnitrite (162 mL, 1.40 mol) in DCM (150 mL) at a rate such that the internal temperature did not exceed 5° C. The yellow solution was stirred at 0° C. for 2 hours and then overnight at room temperature. Warming-up the reaction to room temperature cause slight bubbling, which stops after 15 minutes, at which point the reaction turns to bright orange.

The following morning, the reaction mixture was cooled down to 0° C. and saturated NaHCO₃ was slowly added until no more gas release is observed. The reaction mixture was warmed up to room temperature and stirred for 1 hour, at which point it was poured in water (1 L). Layers were separated and the aqueous layer was extracted with DCM (3×1 L). The combined organic layers were washed with water (1 L). The organic layer was removed, and the aqueous layer containing solid particulates alongside a DCM/water emulsion was filtered on Celite™. The filtrate was transferred into a clean extraction funnel and layers were separated. The combined DCM layers were evaporated in vacuo. The residue was diluted with EtOAc (1 L), washed with water (500 mL) and brine (500 mL), dried over sodium sulfate, filtered and evaporated to a residue which was purified by flash chromatography (SiO₂, 0-30% EtOAc/hexanes) to afford pure Intermediate 3 as a yellow oil (135 g, 52%) and analytical values were found to match previously reported values.

### (iii) Preparation of Intermediate 4 (9-((2R,4S,5R)-4-((tert-butyldimethylsilyl)oxy)-5-(((tert-butyldimethylsilyl)oxy)methyl)tetrahydrofuran-2-yl)-6-vinyl-9H-purine)

Tributylvinyl tin (191.4 mL, 655 mmol) was rapidly added to a degassed mixture of Intermediate 3 (217.95 g, 437 mmol) and (PPh₃)₂PdCl₂ (15.32 g, 21.8 mmol) in anhydrous DMF (2500 mL). The flask was equipped with a reflux condenser and the mixture was heated to 85° C. for 30 minutes after which time it was allowed to cool down, filtered through a Buchner filter containing celite finely grinded with potassium fluoride monohydrate (3:1 Celite™:KF) and the filter cake was washed with EtOAc (500 mL). The filtrate was then filtered again through a Buchner filter containing a mixture of Celite™ and dry silica (~1:1 Celite™:SiO₂) and the filter cake was washed with EtOAc (500 mL). The filtrate was diluted in EtOAc (2000 mL) and washed with saturated NaHCO₃ (1×1500 mL), a 1 M solution of KF (1×10000 mL), water (1×1000 mL) and brine (1×1000 mL), dried over sodium sulfate, and filtered. In order to limit the risks of desired product polymerization, the mixture was concentrated in vacuo until an approximate 2.5 L of EtOAc remained, and this solution containing Intermediate 4 was used directly in the next step. MS calculated for C₂₄H₄₃N₄O₃Si₂ [M+H]+ 491.3, found 491.5.

### (iv) Preparation of Intermediate 5 ((2R,3S,5R)-5-(6-(2-((4-(tert-butyl)phenyl)thio)ethyl)-9H-purin-9-yl)-2-(hydroxymethyl)tetrahydrofuran-3-ol)

Intermediate 4 in ethyl acetate (2.5 L) was treated at room temperature with neat tert-butylthiophenol (90.3 mL, 523 mmol). The mixture was stirred for 60 minutes and was then diluted with EtOAc (2000 ml). It was then washed with 5% Na₂CO₃ (3×1000 mL), water (1×1000 mL) and brine (1000 mL). Sodium sulfate was added to the organic layer and left stirring for 30 minutes before filtering on Celite™. The filter cake was washed with EtOAc and volatiles were removed in vacuo. The residue was dissolved in THE (2000 mL), cooled down to 0° C. and treated with TBAF (1M solution in THF, 961 mL, 961 mmol). The ice bath was removed, and the orange mixture was stirred at room temperature for about 18 hours. Volatiles were concentrated in vacuo and EtOAc (2000 ml) was added. The organic layer was washed with water (2×1000 ml) and brine (1000 ml), treated with sodium sulfate, left to stir for 30 minutes, and filtered through Celite™. The Drying agent was washed with EtOAc (3×350 mL), and volatiles were evaporated in vacuo with DCM co-evaporation, leaving a dark-brown oil of constant mass of 341 g.

The crude compound was diluted in DCM (500 mL) and purified by flash chromatography. Briefly, ~1000 mL of dry silica was treated with hexanes, homogenized, and transferred to a Buchner fritted funnel. The material was purified in two batches, eluting with hexanes (2 CV), 10% MeOH in DCM (2 CV), and 15% MeOH in DCM (3 CV). Evaporation of the collected fractions gave Intermediate 5 (131.18 g, 70% from Intermediate 3 (3 steps)) as dark-brown oil).

¹H-NMR (400 MHz, DMSO-d₆) δ 1.26 (s, 9H), 2.31-2.37 (m, 1H), 2.75-2.82 (m, 1H), 3.37-3.41 (m, 2H), 3.48-3.55 (m, 3H), 3.59-3.65 (m, 1H), 3.87-3.90 (m, 1H), 4.97-5.00 (m, 1H), 5.36 (d, J=4 Hz, 1H), 6.46 (t, J=8 Hz, 2H), 7.29-7.35 (m, 4H), 8.74 (s, 1H), 8.84 (s, 1H). MS calculated for C₂₂H₂₉N₄O₃S [M+H]⁺ 429.2, found 429.4. These values were found to match previously reported values.

### (v) Preparation of Intermediate 6 (2R,3S,5R)-2-((bis(4-methoxyphenyl)(phenyl)methoxy) methyl)-5-(6-(2-((4-(tert-butyl)phenyl)thio)ethyl)-9H-purin-9-yl)tetrahydrofuran-3-ol

Intermediate 5 (9.39 g, 21.9 mmol) was dissolved in anhydrous pyridine (71 mL) and treated with DMAP (134 mg, 1.1 mmol). The orange mixture was cooled down to about −10° C. using a dry ice-water-MeOH bath (15% MeOH) and solid 4,4-dimethoxytriphenylmethyl chloride (DMTr-CI, 9.66 g, 28.5 mmol) was added in three portions over 45 minutes. The light orange reaction mixture was stirred at room temperature for about 18 hours.

MeOH (5 ml) was added, and volatiles were evaporated in vacuo with co-evaporations with toluene and DCM. The crude oil was diluted in DCM containing ~1% Et₃N and the product was dry-loaded onto silica and purified by automated flash chromatography (SiO₂, 60-100% (ethyl acetate+1% Et₃N)/(hexanes+1% Et₃N)) and evaporation of the collected fractions gave Intermediate 6 (7.40 g, 46%) as pale-yellow foam.

¹H-NMR (400 MHz, CDCl₃) δ 1.30 (s, 9H), 2.53-2.59 (m, 1H), 2.85-2.92 (m, 1H), 3.38-3.47 (m, 1H), 3.49-3.54 (m, 2H), 4.17 (br. S, 1H), 4.70-4.73 (m, 1H), 6.50 (t, J=8 Hz), 6.79-6.82 (m, 4H), 7.19-7.41, 13H), 8.18 (s, 1H), 8.80 (s, 1H); ¹³C-NMR (100 MHz, CDCl₃) δ 14.2, 21.0, 31.3, 32.1, 33.3, 34.4, 40.1, 55.2, 60.4, 63.7, 72.63, 84.3, 86.1, 86.6, 113.2, 126.0, 127.0, 127.9, 128.0, 129.9, 130.0, 132.2, 133.4, 135.6, 142.4, 144.5, 149.5, 150.3, 152.3, 158.6, 160.3. MS calculated for C₄₃H₄₇N₄O₅S [M+H]⁺ 731.9, found 731.5. These values were found to match previous literature.

### (vi) Preparation of Precursor X′ ((2R,3S,5R)-2-((bis(4-methoxyphenyl)(phenyl)methoxy) methyl)-5-(6-(2-((4-(tert-butyl)phenyl)thio)ethyl)-9H-purin-9-yl)tetrahydrofuran-3-yl (2-cyanoethyl) diisopropylphosphoramidite)

Intermediate 6 (1.0915 g, 1.49 mmol) was co-evaporated twice with toluene. It was then dissolved in anhydrous DCM (15 mL), flushed with nitrogen, and treated with DIPEA (1.6 mL, 8.96 mmol). The mixture was cooled to 0° C. and 3-((chloro(diisopropylamino)phosphaneyl)oxy)propanenitrile (834 μL, 3.73 mmol) was added dropwise over 1 minute. The mixture was stirred at 0° C. for 1 hour. The resulting mixture was then diluted with DCM containing about 2% Et₃N (50 mL) and transferred to a separatory funnel. The organic layer was washed with saturated aqueous NaHCO₃ (2×30 mL), water (30 mL) and brine (30 mL), dried over sodium sulfate, filtered and concentrated to about 5 mL. This solution was added to a slurry of SiO₂ in DCM (10 mL) and Et₃N (0.5 mL). After evaporation, the compound was purified by automated flash chromatography (SiO₂, 0-30% (ethyl acetate+1% Et₃N)/(hexanes+1% Et₃N)) and evaporation of the collected fractions gave Precursor X′ (1.1549 g, 1.24 mmol, 83%, stored in silanized glass vials, under N₂ atmosphere, and at −20° C.) as a white foam (diastereomeric mixture at the phosphorus center).

¹H-NMR (400 MHz, CDCl₃) δ 1.14 (d, J=4 Hz, 3H), 1.19-1.22 (m, 9H), 1.30 (s, 9H), 1.59-164 (m, 2H), 2.48 (t, J=4H, 1H), 2.63 (t, J=4 Hz, 1H), 2.92-2.94 (m, 1H), 3.33-3.73 (m, 8H), 3.78 (2 overlapping s, 6H), 4.32-4.34 (m, 1H), 4.77-4.79 (m, 1H), 6.48-6.51 (m, 1H), 6.77-6.81 (m, 5H), 7.21-7.41 (m, 12H), 8.21 (s, 0.5H), 8.23 (s, 0.5H), 8.80-8.81 (2 overlapping s, 1H); ³¹P-NMR (243 Hz, CDCl₃) δ 148.7, 148.8. HRMS calculated for C₄₆H₅₁N₅O₇PS [M′+H]⁺ (in situ phosphoramidate hydrolysis product) 848.3247, found 848.3188. These values were found to match previous literature.

### Example 3a—Preparation of Precursor Fmoc-Y′

Precursor Fmoc-Y′ was prepared according to the procedure illustrated in Scheme 2a.

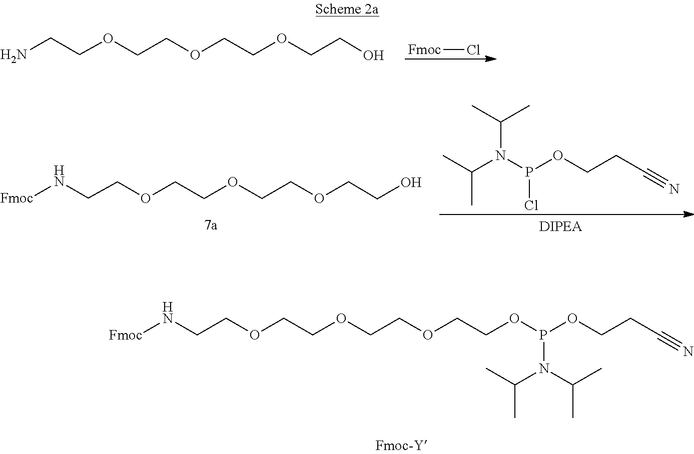

### (i) Preparation of Intermediate 7a ((9H-fluoren-9-yl)methyl (2-(2-(2-(2-hydroxyethoxy)ethoxy)ethoxy)ethyl)carbamate)

2-(2-(2-(2-Aminoethoxy)ethoxy)ethoxy)ethanol (1.00 g, 5.17 mmol) was dissolved in 1,4-dioxane (10 mL) and added to a 0.5 M aqueous solution of NaHCO₃ (20 mL). The mixture was cooled down to 0° C. and a solution of 9-fluorenylmethyl chloroformate (1.473 g, 5.69 mmol) in 1,4-dioxane (10 mL) was added over 5 minutes under vigorous stirring. The mixture was stirred at 0° C. for about 2 hours, allowed to warm up to room temperature and stirred for another approximately 18 hours. The solution was diluted with EtOAc (30 mL), and layers were separated. The organic layer was washed with water (2×15 mL) and brine (15 mL). The organic layer was dried over sodium sulfate, filtered, and concentrated in vacuo. The compound was adsorbed on SiO₂ and was purified by automated flash chromatography (SiO₂, 0-10% MeOH/DCM). Evaporation of the collected fractions gave Intermediate 7a as a colorless oil (5.17 mmol, quantitative) and analytical values were found to match previous literature.

### (ii) Preparation of Precursor Fmoc-Y′ ((9H-fluoren-9-yl)methyl (2-(2-(2-(2-(((2-cyanoethyl) (diisopropylamino)phosphaneyl)oxy)ethoxy)ethoxy)ethoxy)ethyl)carbamate)

Intermediate 7a (1.1763 g, 2.83 mmol) was dissolved in anhydrous DCM (30 mL), flushed with nitrogen, and treated with DIPEA (3.0 mL, 17.00 mmol). The mixture was cooled to 0° C. and 3-((chloro(diisopropylamino)phosphaneyl)oxy)propanenitrile (1.58 mL, 7.08 mmol) was added dropwise over 1 minute. The mixture was stirred at 0° C. for 1 hour and was then diluted with DCM containing about 2% Et₃N (100 mL) and transferred to a separatory funnel. The organic layer was washed with saturated aqueous NaHCO₃ (2×60 mL), water (60 mL) and brine (60 mL), dried over sodium sulfate, filtered, and concentrated to about 5 mL. This solution was added to a slurry of SiO₂ in DCM (10 mL) and Et₃N (0.5 mL). After evaporation, the compound was purified by automated flash chromatography (SiO₂, 0-30% (ethyl acetate+1% Et₃N)/(hexanes+1% Et₃N)) and evaporation of the collected fractions gave Precursor Fmoc-Y′ (1.3336 g, 77%, stored in silanized glass vials, under N₂ atmosphere, and at −20° C.) as a colorless oil.

¹H-NMR (400 MHz, CDCl₃) δ 1.16-1.19 (m, 12H), 1.23-1.29 (m, 2H), 2.63 (t, J=4H, 2H), 3.41 (q, J=4 Hz, 2H), 3.56-3.87 (m, 16H), 4.23 (t, J=4 Hz, 1H), 4.40-4.42 (m, 2H), 5.41-5.44 (m, 1H), 7.32 (dt, J₁<1 Hz, J₂=8 Hz, 2H), 7.41 (t, J=8 Hz, 2H), 7.62 (d, J=8 Hz, 2H), 7.75 (d, J=8 Hz, 2H); ³¹P-NMR (243 Hz, CDCl₃) δ 148.5. MS calculated for C₂₆H₃₄N₂O₈P [M′+H]⁺ (in situ phosphoramidate hydrolysis product) 533.2, found 533.3.

### Example 3b—Preparation of Precursor MTr-Y′

Precursor MTr-Y′ was prepared according to the procedure illustrated in Scheme 2b.

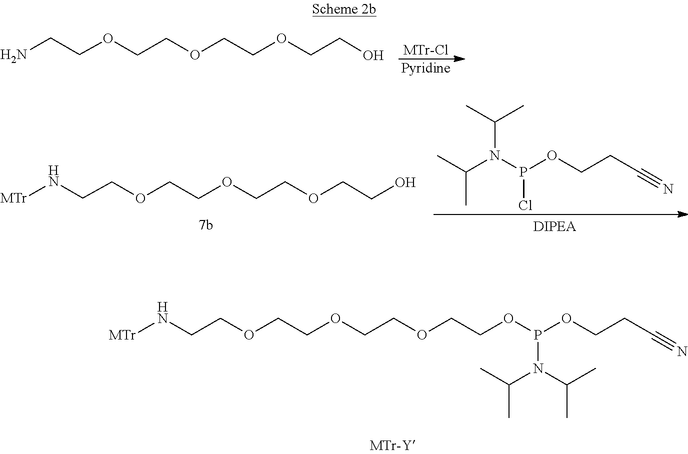

### (i) Preparation of Intermediate 7b ((9H-fluoren-9-yl)methyl (2-(2-(2-(2-hydroxyethoxy)ethoxy)ethoxy)ethyl)carbamate)

2-(2-(2-(2-aminoethoxy)ethoxy)ethoxy)ethan-1-ol (608 mg, 3.14 mmol) was diluted with dry pyridine (7.5 mL). The flask was cooled down to about −10° C. using a ice-water-MeOH bath (15% MeOH) and solid (chloro(4-methoxyphenyl)methylene)dibenzene (922 mg, 2.99 mmol) was added. The mixture was allowed to warm up to room temperature and was stirred for 3 h with TLC monitoring (30% EtOAc/hexanes, revealed by exposure to TFA vapor). Upon completion of the reaction, methanol was added (2 mL) and the mixture was stirred for 30 min. The crude reaction mixture was poured in saturated NaHCO₃ (50 mL) and the mixture was extracted with EtOAc (3×100 mL). The combined organic layers were washed with water (1×100 mL) and brine (1×100 mL), dried over magnesium sulfate, filtered and evaporated in vacuo. The crude oil was purified by automated flash chromatography (Et₃N-deactivated SiO₂, 0-30% (ethyl acetate+1% Et₃N)/(hexanes+1% Et₃N)) and evaporation of the collected fractions gave Intermediate 7b (1.3038 g, 89%, stored in silanized glass vials, under N₂ atmosphere, and at −20° C.) as pale-yellow oil.

¹H-NMR (400 MHz, DMSO-d6) δ 2.11-2.15 (m, 2H), 2.55 (t, J=8 Hz, 1H), 3.36-3.39 (m, 2H), 3.39-3.54 (m, 12H), 3.72 (s, 3H), 4.55 (br. S, 1H), 6.85 (d, J=8 Hz, 2H), 7.16-7.20 (m, 2H), 7.28 (t, J=4 Hz, 6H), 7.39 (d, J=8 Hz, 4H).

¹³H-NMR (100 MHz, DMSO-d6) δ 43.1, 60.2, 69.6, 69.7, 69.8, 70.2, 72.3, 113.0, 126.0, 127.7, 128.2, 129.5, 137.9, 157.4.

### Preparation of Precursor Mtr-Y′ 2-cyanoethyl (1-(4-methoxyphenyl)-1,1-diphenyl-5,8,11-trioxa-2-azatridecan-13-yl) diisopropylphosphoramidite

The same protocol used for the preparation of Fmoc-Y′ was employed with intermediate 7b as starting material (1.93 g, 4.14 mmol). After chromatography (Et₃N-deactivated SiO₂, 0-45% (ethyl acetate+1% Et₃N)/(hexanes+1% Et₃N)), precursor MTr-Y′ (1.98 g, 72%) was obtained as pale-yellow oil.

¹H-NMR (400 MHz, CDCl₃) δ 1.18 (t, J=7 Hz, 12H), 2.05-2.09 (m, 1H), 2.33-2.38 (m, 2H), 2.63 (td, J=2 Hz, 4 Hz, 2H), 3.52-3.54 (m, 2H), 3.58-3.64 (m, 12H), 3.79 (s, 3H), 3.80-3.88 (m, 2H), 6.80-6.82 (m, 2H), 7.16-7.19 (m, 2H), 7.25-7.29 (m, 6H), 7.37-7.39 (m, 2H), 7.46-7.48 (m, 4H); ³¹P-NMR (243 Hz, CDCl₃) δ 148.5. MS calculated for C₂₈H₃₅NO₇P [M′−H]⁻ (in situ phosphoramidite double hydrolysis product) 528.2, found 528.2.

### Example 4—Preparation of Headpieces (HP)

### (i) HP-1 and HP-2/HP-3

In a 2.0 mL microcentrifuge tube, S_HP-1 (SEQ ID NO: 2) or S_HP-2 (SEQ ID NO: 6) (150 μL, 0.727 mM in DNase-free water, 109 nmol) was treated with MMPP (90.1 μL, 2.42 mM in sodium carbonate buffer, 218 nmol) and left to stand for 30 min at room temperature. Sodium hydroxide (43.6 μL, 0.1 M in DNase-free water, 4.36 μmol) was added and the reaction mixture was left to stand at RT for 1 h. A second addition of sodium hydroxide solution (43.6 μL, 0.1 M in Dnase-free water, 4.36 μmol) was made and the reaction mixture was left to stand at RT for 1.5 h. The vial was cooled down to 0° C. and acetic acid (87.2 μL, 0.1 M in Dnase-free water, 8.72 μmol) was added in one portion. The vial was thoroughly vortexed and allowed to stand at 0° C. for 5 minutes and DNA was precipitated (see Example 1).

Briefly, the crude solution (~425 μL) was treated with ~10% V/V of 3 M aqueous sodium acetate containing 0.3 M MgCl₂ (43 μL) followed by 3.5 volumes of cold absolute ethanol (1.5 mL). The resulting cloudy mixture was stirred on a vortexer, and the vial was left at −20° C. for two hours followed by 10 minutes at −80° C. The tube was spun at 5,000 rpm (6 000 g) for 30 minutes and the solid decanted. The walls of the tube were wiped carefully with a Kimwipe™ and the solid was washed with 2 vol. of cold (−20° C.) absolute ethanol (850 μL). The liquid was decanted as before and both liquid washes were recovered in the same vial and kept until the end of the experiment. The pellet at the bottom was dissolved in MES buffer (327 μL, 50 mM, pH 5.52). AS_HP-1 (SEQ ID NO: 1) or AS_HP-2 (SEQ ID NO: 5) (191 μL, 0.626 mM, 120 μmol) was added in one portion to this solution and the vial was incubated at 50° C. for 5 min and then allowed to cool down to 37° C. The vial was incubated at 37° C. for 24 h before being allowed to slowly cool down to room temperature. DNA was precipitated as described above, and the resulting DNA pellet was dissolved in Dnase-free water (110 μL, 1.0 mM nominal DNA concentration). N-Heptylamine solution (152 μL, 5% v/v water:MeCN:DMSO 6:3:1, 51.1 μmol) was added and the vial was gently shaken on an orbital shaker for 1 h at RT. DNA was precipitated as above and the DNA pellet was dissolved DNase-free water (110 μL, 1.0 mM nominal DNA concentration). The solution was transferred to a 96 deep-well plate and the vial was rinsed with DNase-free water (2×100 μL). The crude DNA duplex was purified by preparative IP-HPLC in a single injection and relevant fractions were frozen in dry ice and lyophilized. The content of the fractions was then dissolved in water containing 20% of MeCN, combined and transferred to a single 1.5 mL microcentrifuge tube. The content of the tube was then lyophilized, followed by two more lyophilization from water containing 20% of MeCN to give HP-1 or HP-2 as white fluffy solid which was dissolved in DNase-free water (109 μL) and analyzed.

HP-1 (64 nmol, 59%): OD: 0.591 mM; LCMS (gradient 5) Rₜ=4.51 min, >85% (@260 nm); MS(TOF): calculated deconvoluted mass 16 819, found 16 819; Gel: Polyacrylamide, 15% TBE-Urea, 27 bp.

HP-2 (0.712 μmol, 89±7% (n=4); 0.8 μmol scale): OD: 1.040 mM; LCMS (gradient 2) Rₜ=6.69 min, 95% (@260 nm); MS(Orbitrap™): calculated deconvoluted mass 16 874, found 16 874; Gel: Polyacrylamide, 15% TBE-Urea, 27 bp.

### (ii) AOP-HP-1 and AOP-HP-2/HP-3

In a 1.5 mL microcentrifuge tube, HP-2 (1000 μL, 0.899 mmolar in DNase-free water, 899 nmol) was diluted with Sodium borate buffer (49.7 μL, 250 mmolar, pH=9.5, 12.4 μmol). The vial was cooled down to 0° C. and treated with Fmoc-AOP-acid (357 μL, 200 mM in DMA, 71.4 μmol) followed by DMT-MM (335 μL, 200.0 mM in DNase-free water, 66.9 μmol). The vial was allowed to stand at 4° C. for 2 h followed by a second treatment of Fmoc-AOP-acid (357 μL, 200 mM in DMA, 71.4 μmol) and DMT-MM (335 μL, 200.0 mM in DNase-free water, 66.9 μmol). The vial was vortexed and left to stand at 4° C. for ca 18 h. DNA was precipitated, and the white solid obtained was dissolved in 900.0 μL of DNase-free water. Piperidine (100 μL, 1.01 mmol, 10% final concentration) was added, the mixture was stirred well on a vortexer (the solution eventually became cloudy), and the vial was allowed to stand at room temperature for 1 h. DNase-free water (1000 μL) was added, and DNA was precipitated using 0.1 V of 5 M NaOAc containing 1 M NaCl and 4 V of absolute ethanol. The crude product was dissolved in 1 mL of TEAA buffer (100 mM, pH=7.5) and purified by preparative HPLC using a gradient of 8-18 B/A over 15 min. Pure fractions were combined and lyophilized a total of 4 times with resuspension in 20% MeCN/H₂O in between each one to remove any residual buffer, and the purified DNA was dissolved in DNase-free water (1000 μL) and analyzed. AOP-HP-1 was synthetized in a similar fashion.

AOP-HP-2 (0.801 μmol, 89%): OD: 0.899 mM; HPLC (gradient 2) Rₜ=6.78 min, 84% (@260 nm); MS(Orbitrap): calculated deconvoluted mass 17 121, found 17 120.

AOP-HP-1 (10.0 nmol, 71% [14 nmol scale]): OD: 0.332 mM; HPLC (gradient 2) Rₜ=6.95 min, 69% (@260 nm); MS(TOF): calculated deconvoluted mass 17 066, found 17 066.

(iii) AzOP-HP-2/HP-3

The same procedure used for the synthesis of AOP-HP-2 was employed on a 10 nmol scale, with 1-azido-3,6,9,12-tetraoxapentadecan-15-oic acid as the coupling partner. DNA precipitation using 3M NaOAc containing 0.3M MgCl₂ yielded the crude product, which was dissolved in Dnase-free water (50 μL) and analyzed, without further purification.

AzOP-HP-2 (10.7 nmol, 107%): OD: 0.535 mM; HPLC (gradient 2) Rₜ=6.40 min; MS(Orbitrap): calculated deconvoluted mass 17 147, found 17 147.

(iii) AOP-HP-2-T1

In a 2.0 mL Eppendorff, AOP-HP-2 (61.96 μL, 0.807 mmolar in DNase-free water, 50.0 nmol) was diluted with DNAse-free water (75.00 μL). The resulting solution was treated with Tag Cycle 1_1 (75.00 μL, 1.00 mmolar in DNase-free water, 75.0 nmol) and 10× Buffer (23.78 μL), vortexed 1 minute, and then treated with T-4 enzyme (2.0 μL, 1 U/uL in water), vortexed 1 minutes and gently shake overnight at room temperature. The crude solution was precipitated (24 μL (3M NaOAc)+960 μL absolute ethanol. The crude DNA pellet was resuspended in DNase-free water (100 μL) and purified by preparative IP-HPLC and relevant fractions were frozen in dry ice and lyophilized. The content of the fractions was then dissolved in water containing 20% of MeCN, combined and transferred to a single 1.5 mL microcentrifuge tube. The content of the tube was then lyophilized, followed by two more lyophilization from water containing 20% of MeCN to afford the product as white fluffy solid which was dissolved in DNase-free water (20.0 μL) and analyzed.

AOP-HP-2-T1 (30.0±3.2 nmol, 60±6%, n=3): OD: 0.600±0.064 mM; HPLC (gradient 2) Rₜ=7.60 min; MS(Orbitrap): calculated deconvoluted mass 22 691, found 11 345 (M/2); Gel: Agarose, 40 bp

AOP-HP-A-P1-T1 (29.9±4.4 nmol, 60±9%, n=3): OD: 0.598±0.087 mM; HPLC (gradient 2) Rₜ=5.93 min; MS(Orbitrap): calculated deconvoluted mass 17 606, found 17 605; Gel: Agarose, 35 bp.

HP-3 (AS_HP-3 (SEQ ID NO: 7) and S_HP-3 (SEQ ID NO: 8)) was synthesized using the same method as for HP-2.

### Example 5—HP Characterization

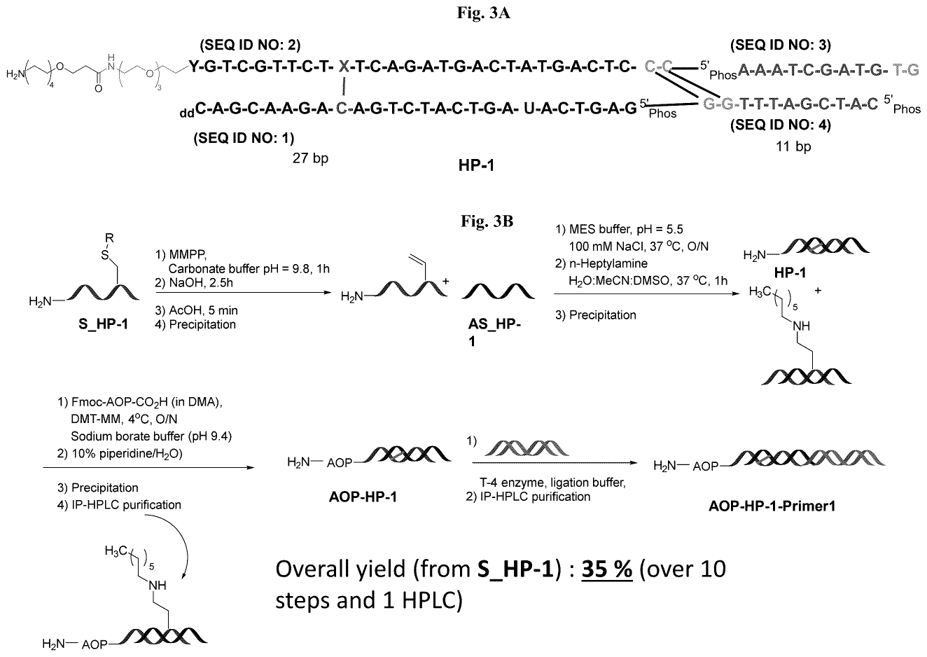

With the relevant complementary oligonucleotides in hand, we set on to explore various primer ligation conditions based on the previously published literature. Best results were obtained when the oxidation step was performed using Magnesium Monoperoxyphtalate (MMPP) in basic (pH=9.8) carbonate buffer, followed by NaOH-mediated elimination (FIG. 3B). Since the following conjugation step needs to be performed under acidic media, neutralization of excess base with dilute acetic acid prior to ethanol precipitation led to improved conjugation yields. Following precipitation and reconstitution of the oxidized and eliminated S_HP-1 in the crosslinking buffer (50 mM MES, pH=5.5, 100 mM NaCl), the complementary AS_HP-1 (SEQ ID NO: 1) is added and the mixture is heated to 80° C. for 5 minutes to break unwanted oligomers, followed by overnight incubation at 37° C. to afford the desired crosslinked product.

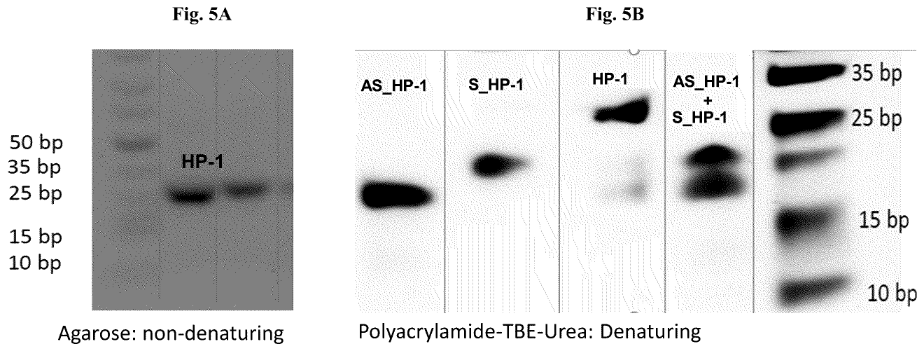

Since the conjugation reaction cannot be pushed to full completion, a residual amount of the conjugated alkene starting material annealed to the complementary AS_HP-1 (SEQ ID NO: 1) remains in the mixture, and considering that this double-stranded oligonucleotide is challenging to separate from the desired product by HPLC, a treatment with a concentrated solution of N-heptylamine is made to ensure complete conversion of the residual alkene to the corresponding long chain amine prior to salt exchange and precipitation. Due to the presence of a seven-carbon chain, ds(Side Product)_C7 can easily be separated from the desired product by preparative ion-pairing HPLC (IP-HPLC), and the desired HP can be obtained in good purity and yield (over 8 steps). The inter-strand crosslinking was confirmed by denaturing 15% TBE-Urea polyacrylamide gel electrophoresis (PAGE). It is important to note that overoxidation to the sulfone, which cannot undergo base promoted elimination, reducing the overall yield, is a significant problem that needs to be carefully monitored trough LCMS. The use of alternative oxidants was unsuccessful at circumventing the issue, therefore careful control of the reaction conditions must be employed, with reaction temperature, time, buffer composition and number of equivalents of the oxidizing agent all playing a role in avoiding the overoxidation side product formation. The structure of HP-1 was ascertained by LC-MS and both denaturing and non-denaturing gel electrophoresis (FIGS. 5A and 5B).

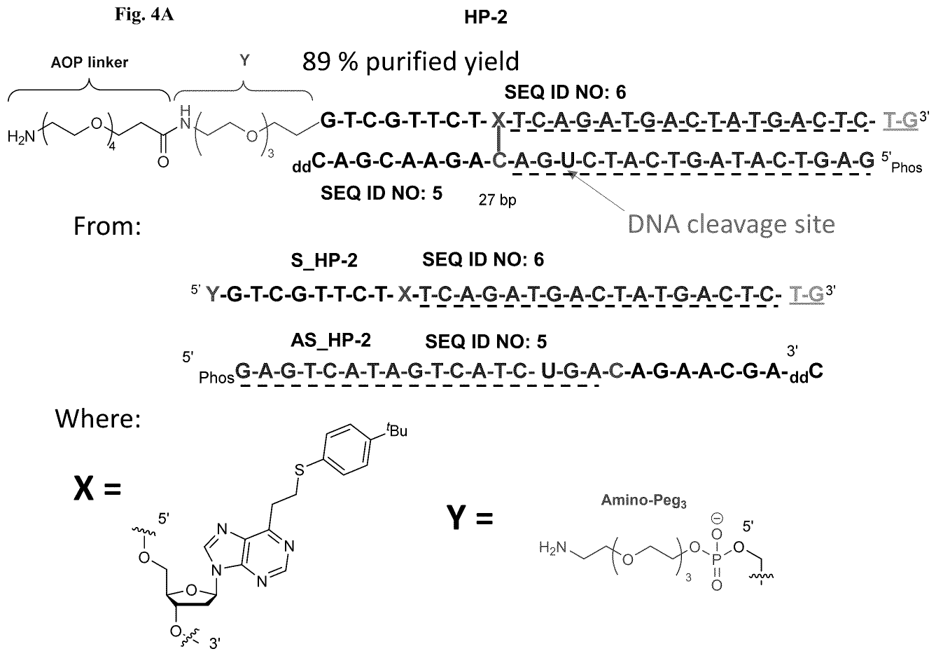

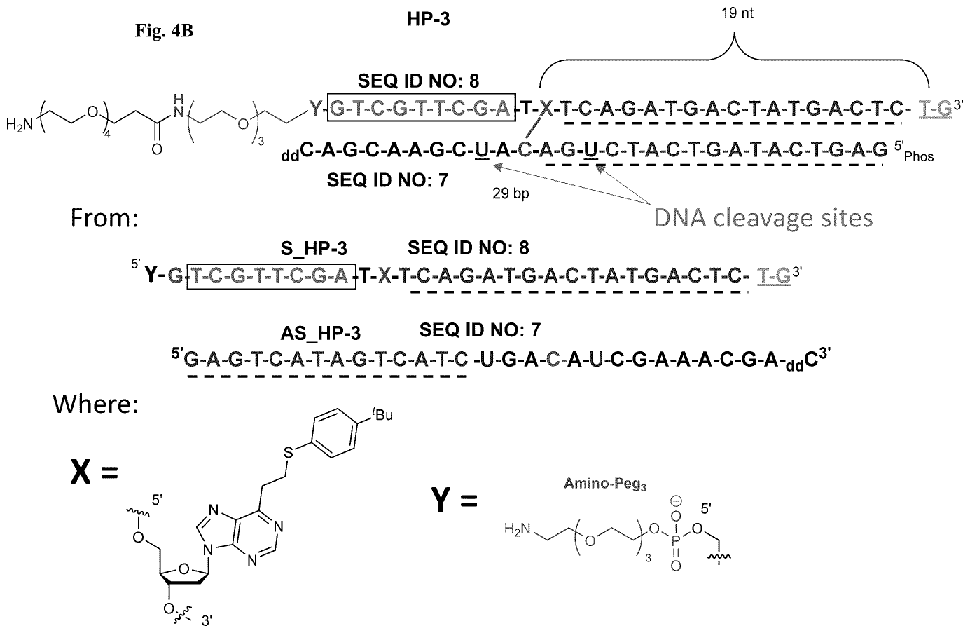

In order to obtain a headpiece assembly that retains the same distance between the coding DNA and the pharmacophore as the HP described in WO2005058479A2 (HP-A), an additional coupling using 1-amino-(PEG)₄ acid as the coupling partner was performed, giving AOP-HP-1, which is ready for pharmacophore attachment. It is important to note that including those four additional PEG units in precursor Y led to significant decomposition of the material, therefore the attachment was performed after headpiece assembly. Alternatively, coupling with 1-azido (PEG)₃ acid can be done to obtain an azide-functionalized HP. In order to use the headpiece in a library setting, an enzymatic ligation with a PCR forward primer needs to be performed, yielding AOP-HP-1-Primer1, and the latter assembly can be use directly for library synthesis (FIGS. 3A and 3B). HP-2 (FIG. 4A) and HP-3 (FIG. 4B) which have the primer integrated within the sequence, function similarly to HP-1 with the added benefit of avoiding the additional step of primer coupling, and therefore can be directly used in the DELs.

### Example 6—Comparative Evaluation of the HP Described Herein with Existing HPs

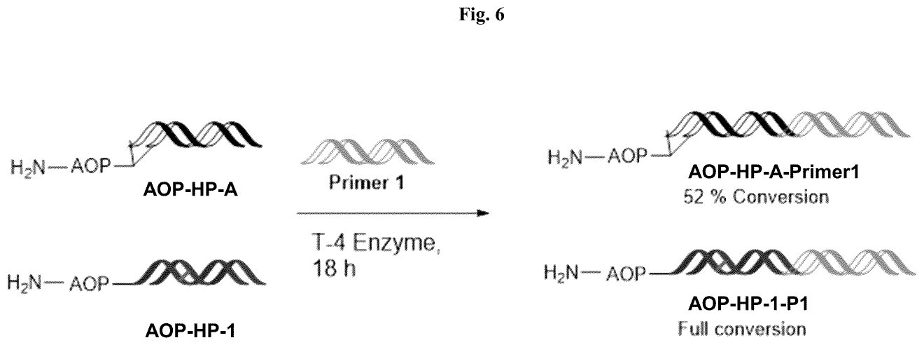

To quantify advantages of HP described herein over existing technologies, the enzymatic ligation efficiency of DNA codons was evaluated using the HP described in WO2005058479A2 (HP-A). Under standard coupling conditions, AOP-HP-1 led to full conversion, whereas AOP-HP-A only gave 52% conversion (FIG. 6; Table 2). While performing a second ligation cycle on AOP-HP-A allowed complete conversion, these results strongly suggest that enzymatic ligation on HP-1 perform more efficiently. This could be explained by both the longer DNA sequence of HP-1 and the stronger strand association brought on by the covalent linkage. Shorter oligonucleotides can lead to oligomerization which impairs the ligation process. Moreover, having a stronger strand association may help reduce the formation of secondary structures in the DNA, which can also be detrimental to ligation yields. Having to perform a second ligation step, besides wasting potentially costly material, may also negatively impact the purity of the product, as shown by the lower post-purification purity of AOP-HP-A-P1 compared with AOP-HP-1-P1 (Table 2).

TABLE 2
Comparative data for ligation of AOP-HP-A and of AOP-HP-1

| | | | Purified | Final |
|---|---|---|---|---|
| | Conversion % - | Conversion % - | yield | purity |
| Substrate | 1^{st} ligation | 2^{nd} ligation | (%) | (%, 260 nm) |
| AOP-HP-A | 52 | 100 | 54 | 56 |
| AOP-HP-1 | 100 | N/A | 53 | 82 |

To demonstrate a gain of chemical stability brought on by the interstrand crosslink, HP-2 was compared to either HP-A and/or HP-A-P1 under a variety of highly strenuous conditions. Stability of headpieces under acidic buffers, various metal catalysts typically used in DEL synthesis and a water-stable Lewis acid was ascertained by LCMS analysis. The crude precipitated mixtures were spiked with an internal standard (AS_HP-2 was used for that purpose) and analyzed in HILIC mode. For each condition, the UV ratios of the headpiece and internal standard were compared to those of a control sample of the same composition but that didn't undergo heating (t=0), and the results are expressed as percentages of remaining DNA compared to t=0. The results are summarized in Table 3, where it is apparent that HP-2 provides superior resistance to acids, Pd, Cu(I), and Cerium triflate. Since Pd, Cu and Ce are typically interacting with DNA trough the nucleobases, causing the formation of DNA-metal clusters, modifications of the bases (mainly deamination), depurinations and 5′-Phosphate hydrolysis (at acidic pH) and double strand breaks. All these side reactions compromise DNA integrity, resulting in lower yields and impaired DNA amplifiability. The fact that AOP-HP-2 resists better to strenuous conditions involving acids (acidic buffer and Lewis's acid) and metals (Pd, Cu) supports our hypothesis that increased strand association by way of internal crosslinking results in improved chemical stability.

TABLE 3
Comparative chemical resistance data of AOP-HP-2 and AOP-HP-A and/or AOP-HP-A-
P1

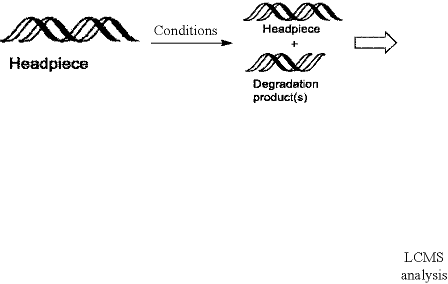

| Entry | Headpiece | Conditions | DNA remaining (%) |
|---|---|---|---|
| 1 | AOP-HP2 | MES buffer (50 mM, pH 5.0), | 52 |
| | AOP-HP-A | 95° C., 3 h | 0 |
| 2 | AOP-HP2 | Tris-HCl buffer (10 mM, pH 8.5), | 100 |
| | AOP-HP-A | 95° C., 45 min | 66 |
| 3 | AOP-HP2 | 20 eq. Pd(OAc)₂, Na- | 60 |
| | AOP-HP-A-P1 | Phaosphate buffer (250 mM, | 18 |
| | AOP-HP-A | pH 6.5), 75° C., 1 h | 21 |
| 4 | AOP-HP2 | 20 eq. tBuXPhosPd GenIII, Na- | 58 |
| | AOP-HP-A-P1 | Phaosphate buffer (250 mM, | 27 |
| | AOP-HP-A | pH 6.5), 75° C., 1 h | 24 |
| 5 | AOP-HP2 | 20 eq. CuI, Na-Phaosphate | 97 |
| | AOP-HP-A-P1 | buffer (250 mM, pH 6.5), 75° C., | 47 |
| | AOP-HP-A | 16 h | 75 |
| 6 | AOP-HP2 | 200 eq. Ce(OTf)₄, H₂O, MeCN, | 40 |
| | AOP-HP-A-P1 | 75° C., 5 h | 17 |
| | AOP-HP-A | | 0 |

In a third series of experiments, we set out to evaluate the performance of HP-2 in real reaction setting, with the goal of defining the scope of possible DNA-supported reactions that can be performed using this technology. To that end, a variety of chemical transformation were tested, using a previously reported multistep methodology that can deliver values for both the chemical yield and the DNA damage endured during the reaction (in selected examples). This value for the remaining % of amplifiable DNA is of particular importance, since certain transformation are known to destroy more than half of the amplifiable DNA, resulting in libraries that are more difficult to amplify and decode since these damaged DNA are often inseparable from the library. First, amide formation reactions, the most often used reaction in DELs, were examined, either using DMT-MM or HATU couplings using an acid or through direct acylation with a succinimide ester (Table 4). As can be observed, HP-2 was found to yield to superior (entries 1, 2, 4, 5) or equal (entries 3) DNA recovery compared to HP-A-P1 (or HP-A, when measured). In certain cases (entry 4), conversion to the desired product was also improved for HP-A supported pharmacophores.

TABLE 4
Comparative performance data of different DNA headpieces under various amide
formation conditions.

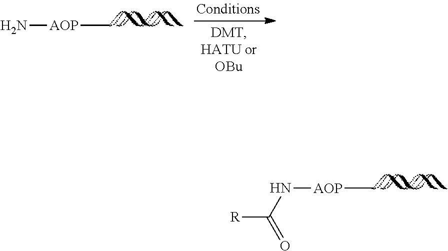

| Entry | Headpiece | Diversity Element | Conditions | DNA Recovery (%) | Conversion (%) |
|---|---|---|---|---|---|
| 1 | AOP-HP2 AOP-HP-A | 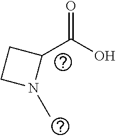 n = 2 | DMT: Acid, DMT- MM, Na-Borate (pH 9.4), 4° C. | 95 93 | 100 100 |
| 2 | AOP-HP2 AOP-HP-A-P1 AOP-HP-A | 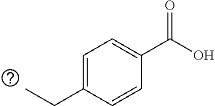 n = 3 | | 74 57 56 | 100 100 100 |
| 3 | AOP-HP2 AOP-HP-A-P1 | 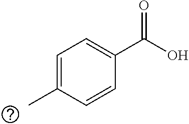 n = 2 | HATU: Acid, HATU, DIPEA, Na- Borate (pH 9.4), RT | 96 96 | 99 99 |
| 4 | AOP-HP2 AOP-HP-A-P1 AOP-HP-A | 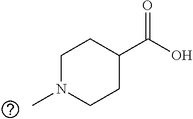 n = 2 | | 100 94 92 | 100 91 91 |
| 5 | AOP-HP2 AOP-HP-A-P1 | 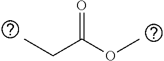 [symbol] n = 1 | OSu: ROSu, Na- Borate (pH 9.4), 4° C. | 100 88 | 95 95 |

[symbol]

indicates data missing or illegible when filed

Next, we evaluated various deprotection and diversification reactions (Table 5), particularly reactions involving the use of transition metal catalysts and low pH.

TABLE 5
Comparative performance data of various DNA headpieces under deprotections, and
metal-catalyzed diversification reactions

| | | | | | |
|---|---|---|---|---|---|
| Entry | Headpiece | Diversity Element | DNA Recovery (%) | Conversion (%) | Purified ligation yield (%) |

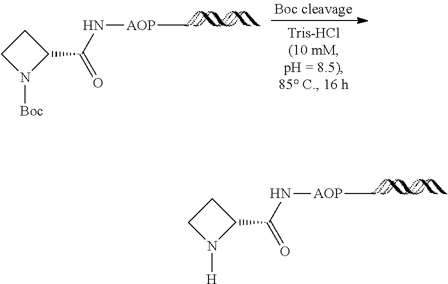

| | | | | | |
|---|---|---|---|---|---|
| 1 | AOP-HP2 | N/A | 50 | quant. | Not performed |
| | AOP-HP-A | | 34 | quant. | |

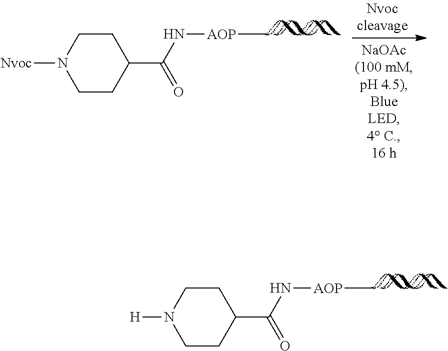

| | | | | | |
|---|---|---|---|---|---|
| 2 | AOP-HP2 | N/A | 92 | quant. | Not performed |
| | AOP-HP-A | | 80 | quant. | |

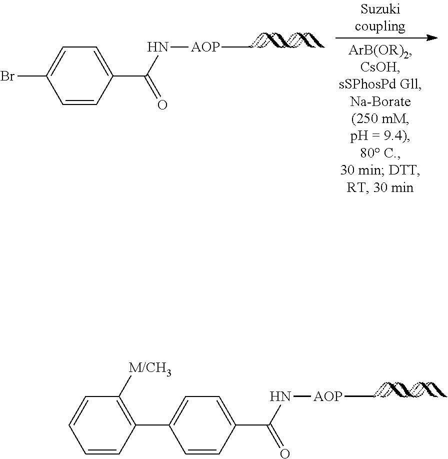

| | | | | | |
|---|---|---|---|---|---|
| 3 | AOP-HP2 | o-methylphenylboronic acid | 82 | quant. | Not performed |
| | AOP-HP-A-P1 | | 80 | quant. | |
| 4 | AOP-HP-2-T1 | phenylboronic acid | 94 | quant. | Not performed |
| | AOP-HP-P1-T1 | | 96 | quant. | |

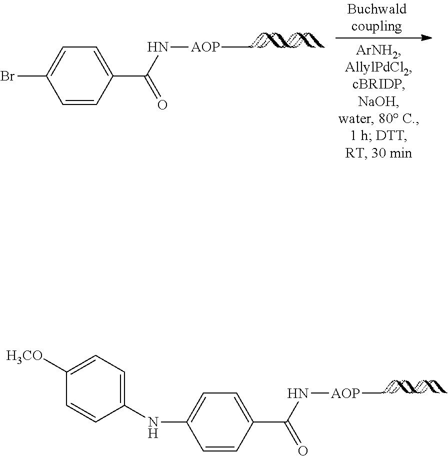

| | | | | | |
|---|---|---|---|---|---|
| 5 | AOP-HP2-T1 | p-methoxyaniline | 100 | quant. | 27 + 4* (n = 3) |
| | AOP-HP-A-P1-T1 | | 74 | quant. | 12 + 5* (n = 3) |

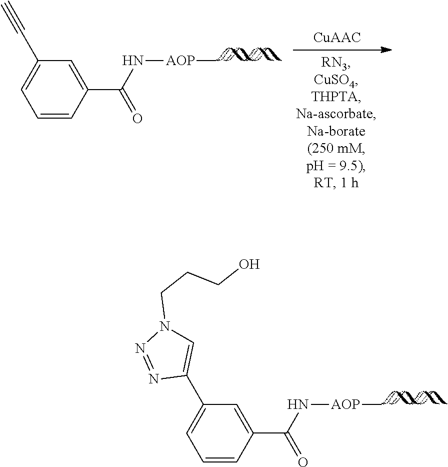

| | | | | | |
|---|---|---|---|---|---|
| 6 | AOP-HP2-T1 | 3-azidopropanol | 100 | quant. | 52 ± 6 (n = 3) |
| | AOP-HP-A-P1-T1 | 3-azidopropanol | 100 | quant. | 50 ± 12 (n = 3) |

AOP-HP-2 was shown to perform similarly or better than the conventional technology in all cases examined, with advantages in terms of DNA recovery found for reactions resulting in more difficult precipitations, such as Boc cleavages (requiring a modified precipitation protocol due to the presence of residual Tris, entry 1) or Buchwald reactions (whose reaction conditions tend to favor the formation of Pd-DNA clusters, entry 5). This observation is in accordance with the data presented in Table 2, where DNA recovery of AOP-HP-2 were generally higher, and in Table 3, where AOP-HP-2 demonstrated better resistance to Pd-induced degradation. In the case of the Buchwald reaction (entry 5), subsequent enzymatic ligation of a mock DNA tag followed by IP-HPLC purification gave significantly (p<0.05) higher yield on AOP-HP-2 than on the conventional counterpart (AOP-HP-A), due to a combination of higher precipitation yields and more efficient purification due to cleaner crude purity for HP-2 supported molecules (For comparison, purified ligation yields for headpieces AOP-HP2-P1-T1 and AOP-HP-A-P1-T1 that didn't undergo metal catalyzed reaction are 69 and 66% respectively). Moreover, purified DNA from Buchwald and CuAAC reactions was shown to be fully amplifiable by qPCR for both headpieces compared (AOP-HP-2 and AOP-HP-A), which indicates that the purification process was effective.

The ability of DNA anchored trough HP-2 and HP-A to be amplified by PCR enzymes was quantified using qPCR on triplicate sample (Table 6, entries 1 and 2). HP-2 was shown to amplify moderately better than HP-A, using Cr counts as measure for the amplifiability of DNA solution of identical concentration. The effect was found to be statistically significant (p<0.01) and may provide advantages for the detection of low-concentration entities anchored on HP-2 in the context of pulldown assays.

TABLE 6
Comparative qPCR amplification results
for AOP-HP-1/2 and AOP-HP-A.

| | | | Standard | |
|---|---|---|---|---|
| | | Cₜ | deviation | |
| | | (mean of | (triplicate | |
| Entry | Headpiece | n = 2) | samples) | p value |
| 1 | AOP-HP-2-T1-T2-CP | 7.0 | 0.1 | 0.0051 |
| 2 | AOP-HP-A-P1-T1-T2-CP | 7.6 | 0.1 | |

| | | | |
|---|---|---|---|
| 3 | Vmb1-HP-1-P1-T1-T2-T3- | 8.0 | N/A |
| | T4-CP | | |
| 4 | Vmb1-HP-A-P1-T1-T2-T3- | 9.4 | |
| | T4-CP | | |

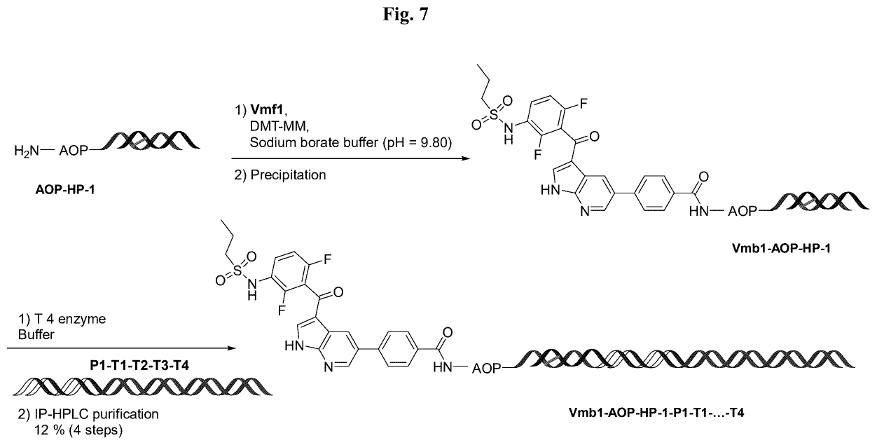

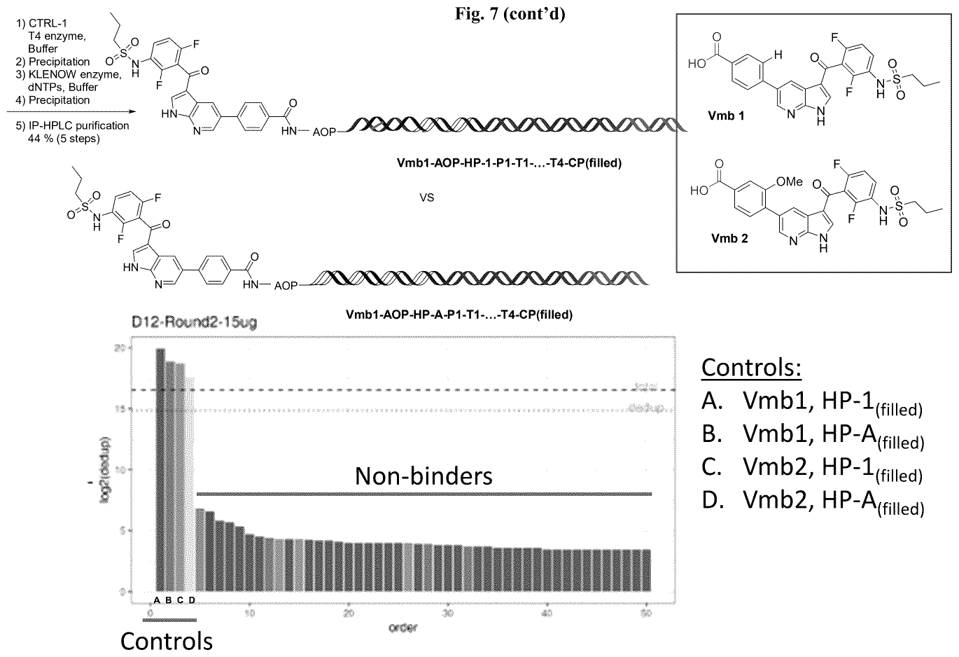

Finally, in order to validate that the present HP technology can be used effectively in the context of a pulldown assay, a Vemurafenib (Vmb1) derivative has been anchored to AOP-HP-1 using standard DMT-MM coupling conditions forming an amide bond between the amine group on the AOP moiety and a carboxylic acid replacing the chlorine atom on the Vemurafenib molecule, followed by enzymatic ligation of a full-length DNA fragment containing the forward primer (Primer 1) and four tags of 9 bp each to represent four synthetic cycles (FIG. 7). This latter step performed poorly, presumably due to the low quality of the P1- . . . -T4 oligonucleotide. The closing primer, which contains a degenerative sequence of 14 nt that can help quantify binders, is added after HPLC purification, and the degenerative sequence is filled-in by treatment with KLENOW™ enzyme in presence of all four deoxy-Nucleosides triphosphate (dNTPs), before the compound is purified one last time by IP-HPLC.

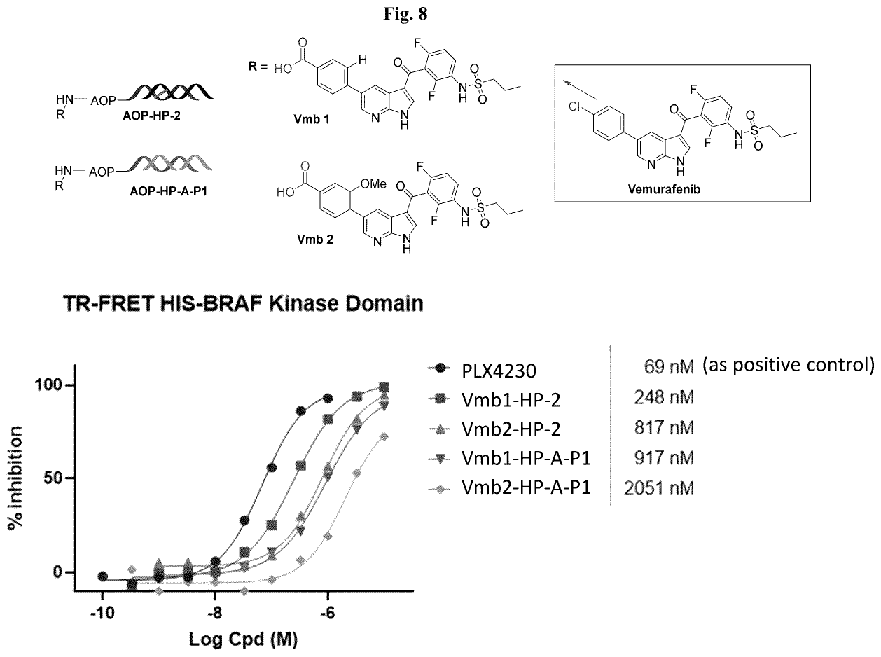

Binding affinities of AOP-HP-2-, and AOP-HP-A-P1-supported vemurafenib derivatives Vmb1 and Vmb2 towards BRAF was ascertained using a TR-FRET assay (FIG. 8), with the use of shorter DNA sequences justified by the amount of material required. In both pharmacophores' studies, AOP-HP-2-supported molecules had higher binding affinities (248 nM vs 917 nM and 817 nM vs 2051 nM), possibly due to the reduced steric bulk of the single-stranded PEG linker used in HP-2 compared to the double-stranded PEG linker of HP-A.

DNA readability of Vmb1-AOP-HP-1-P1-T1-T2-T3-T4-CP was also established (Table 6, entry 3) and compared to an identical control synthetized using HP-A (Vmb1-AOP-HP-1-P1-T1-T2-T3-T4-CP, Table 6, entry 4). In this setting, the control synthesized using AOP-HP-1 was also shown to amplify more easily than its AOP-HP-A counterpart. A small 72-member DEL comprised of BRAF nonbinders was spiked with Vmb1-AOP-HP-1-P1-T1-T2-T3-T4-CP, and subjected to a mock pulldown assay using His-tagged BRAF as the protein of interest. Vmb1-AOP-HP-1-P1-T1-T2-T3-T4-CP could still be readily amplified with high sequence fidelity after two rounds of selection (using fresh protein each time).

### Experimental Procedures for Example 6

### (i) Headpiece Stability Comparison Under Strenuous Conditions (Table 3)

AOP-HP-2 was compared to AOP-HP-A, and AOP-HP-A-P1 (some examples). For each entry, headpieces (2-5 nmol scale) were treated with the reported condition (metals were added as 20 mM solutions in DMSO and Cerium triflate as a 200 mM solution in MeCN) in at least two replicates. One replicate per headpiece/condition was immediately quenched with the appropriate quench solution (see below) and precipitated and served as a reference (t=0), while the remaining samples were incubated at the reported temperature for the reported duration before being quenched with the appropriate quench solution (200 eq) and precipitated. All samples were diluted with Dase-free water, and LCMS samples were prepared by taking 2 μL of the crude solution, and dilution it in 8 μL of DNase-free water containing AS_HP-2 as an internal standard. MeCN (10 uL, final concentration 50%) was added and samples were analyzed by LCMS (Waters BEH Amide column (2.5 microns, 2.1×100 mm) and solvents A: 75:25 H₂O/MeCN, 8 mM NH₄OAc, 5 μM Medronic acid and B: 25:75 H₂O/MeCN, 8 mM NH₄OAc, 5 μM Medronic acid, gradient of 45-90% A/B, UV monitoring at 260 nm, Flow diverted from 0-5 and 12-15 min, ThermoFisher™ Q-Exactive mass spectrometer).

Analysis was done as follow: all samples for which the headpiece mass was no longer detected were assigned the value “0”. For those where the headpieces were present, the UV ratios Headpieces/Standard were generated, and reported as percentages of t=0.

### Quench Solutions:

Buffer (entries 1,2): N/A (Dnase-free water)
Pd (entries 3,4): Sodium diethyldithiocarbamate, 50 mM in Dnase-free water
Cu (entry 5): DTT, 50 mM in Dnase-free water
Lewis Acid (entry 6): NaOH, 1 M in Dnase-free water

### (ii) Headpiece Performance Comparison Under Amide Formation Conditions (Table 4)

### a. ((R)-azetidine-2-carbonyl)-AOP-HP (Entry 1)

In a 1.5 mL Eppendorf tube, AOP-HP-2 (25.0 μL, 0.807 mM in Dnase-free water, 20 nmol) was diluted in sodium borate buffer (40 μL, 250 mmolar, pH=9.5, 10 μmol), treated with N-Boc-(R)-azetidine-2-carboxylic acid (8.0 μL, 200 mM in DMA, 1.6 μmol) and DMT-MM (7.5 μL, 200 mM in DNase-free water, 1.5 μmol). The reaction was thoroughly mixed and left to stand 2 h at 5 0° C. A second addition of N-Boc-(R)-azetidine-2-carboxylic acid (8.0 μL, 200 mM in DMA, 1.6 μmol) and DMT-MM (7.5 μL, 200 mM in DNase-free water, 1.5 μmol) was made. The reaction was thoroughly mixed and left to stand 20 h at 4° C. The reaction mixture was precipitated (7.5 μL (3M NaOAc+0.3M MgCl₂)+300 μL absolute ethanol). The crude DNA pellet was resuspended in DNase-free water (20.0 μL) and analyzed.

(N-Boc-(R)-azetidine-2-carbonyl)-AOP-HP-2 (18.90 nmol, 95%): OD: 0.945 mM; HPLC (HILIC) Rₜ=7.69 min; MS(Orbitrap): calculated deconvoluted mass 17 304, found 17 304.

(N-Boc-(R)-azetidine-2-carbonyl)-AOP-HP-A (18.63 nmol, 93%): OD: 0.932 mM; HPLC (HILIC) Rₜ=5.92 min; MS(Orbitrap): calculated deconvoluted mass 5 368, found 5 367.

### b. 4-(hydroxymethyl)benzoyl-AOP-HP (Entry 2)

In a 1.5 mL Eppendorf tube, AOP-HP-2 (24.8 μL, 0.807 mM in Dnase-free water, 20 nmol) was diluted in sodium borate buffer (40 μL, 250 mmolar, pH=9.5, 10 μmol), treated with 4-(hydroxymethyl)benzoic acid (8.0 μL, 200 mM in DMA, 1.6 μmol) and DMT-MM (7.5 μL, 200 mM in Dnase-free water, 1.5 μmol). The reaction was thoroughly mixed and left to stand 2 h at 5° C. A second addition of 4-(hydroxymethyl)benzoic acid (8.0 μL, 200 mM in DMA, 1.6 μmol) and DMT-MM (7.5 μL, 200 mM in Dnase-free water, 1.5 μmol) was made. The reaction was thoroughly mixed and left to stand 20 h at 5° C. The reaction mixture was precipitated (7.5 μL (3M NaOAc+0.3M MgCl₂)+300 μL absolute ethanol). The crude DNA pellet was resuspended in Dnase-free water (15.0 μL) and analyzed.

4-(hydroxymethyl)benzoyl-AOP-HP-2 (14.84 nmol, 74%): OD: 0.742 mM; HPLC (gradient 2) Rₜ=7.97 min; MS(Orbitrap): calculated deconvoluted mass 17 255, found 17 256.

4-(hydroxymethyl)benzoyl-AOP-HP-A-P1 (11.39 nmol, 57%): OD: 0.569 mM; HPLC (gradient 2) Rₜ=6.90 min; MS(Orbitrap): calculated deconvoluted mass 12 170, found 12 170.

4-(hydroxymethyl)benzoyl-AOP-HP-A (11.14 nmol, 56%): OD: 0.643 mM; HPLC (gradient 2) Rₜ=7.53 min; MS(Orbitrap): calculated deconvoluted mass 5 319, found 5 318.

### c. 4-bromobenzoyl-AOP-HP (Entry 3)

In a 1.5 mL Eppendorf tube, AOP-HP-2 (2.2 μL, 0.811 mM in Dnase-free water, 1.76 nmol) was diluted in sodium borate buffer (5.5 μL, 150 mmolar, pH=9.5, 0.82 μmol) and water (2.5 μL). In a separate 0.5 Eppendorf tube, HATU (1.3 μL, 200 mM in (3:1) MeCN/DMSO, 0.30 μmol), DIPEA (1.5 μL, 200 mM in (3:1) MeCN/DMSO, 0.34 μmol) and 4-bromobenzoic acid (1.4 μL, 200 mM in (3:1) MeCN/DMSO, 0.32 μmol) were combined and vortexed. The vial was left to stand at 4° C. for 5 min before being added to the DNA solution. The reaction vial was vortexed and left to stand at RT for 1 h, after which a second addition of HATU, DIPEA and 4-bromobenzoic acid (preactivated 5 min at 4° C.) was made and the vial was left to stand for 2 h. The reaction mixture was diluted with 1 volume of Dnase-free water (~20 μL) and precipitated (4.0 μL (3M NaOAc+0.3M MgCl₂)+160 μL absolute ethanol). The crude DNA pellet was resuspended in Dnase-free water (20.0 μL) and analyzed.

4-bromobenzoyl-AOP-HP-2 (1.70 nmol, 96%): OD: 0.085 mM; HPLC (gradient 3) Rₜ=8.98 min; MS(Orbitrap): calculated deconvoluted mass 17 303, found 17 304.

4-bromobenzoyl-AOP-HP-A-P1 (1.92 nmol, 96%, 2.0 nmol scale): OD: 0.096 mM; HPLC (gradient 3) Rₜ=7.97 min; MS(Orbitrap): calculated deconvoluted mass 12 217, found 12 218.

### d. (N-Nvoc-piperidine-4-carbonyl)-AOP-HP (Entry 4)

The protocol used for the synthesis of 4-bromobenzoyl-AOP-HP-2 was used with headpieces AOP-HP-2 (2.5 μL, 0.811 mM in Dnase-free water, 2.0 nmol), AOP-HP-A-P1 (2.0 μL, 1.010 mM in Dnase-free water, 2.0 nmol) and AOP-HP-A (1.7 μL, 1.146 mM in Dnase-free water, 2.0 nmol) as starting material and 1-(((4,5-dimethoxy-2-nitrobenzyl)oxy)carbonyl)piperidine-4-carboxylic acid (2×1.6 μL, 200 mM in (3:1) MeCN/DMSO, 2×0.32 μmol) as the donor acid.

(N-Nvoc-piperidine-4-carbonyl)-AOP-HP-2 (2.00 nmol, 100%): OD: 0.202 mM; HPLC (gradient 3) Rₜ=9.89 min; MS(TOF): calculated deconvoluted mass 17 471, found 17 471.

(N-Nvoc-piperidine-4-carbonyl)-AOP-HP-A-P1 (1.88 nmol, 94%): OD: 0.188 mM; HPLC (gradient 3) Rₜ=8.92 min; MS(TOF): calculated deconvoluted mass 12 385, found 12 386.

(N-Nvoc-piperidine-4-carbonyl)-AOP-HP-A (1.71 nmol, 91%): OD: 0.171 mM; HPLC (gradient 3) Rₜ=9.55 min; MS(TOF): calculated deconvoluted mass 5 534, found 5 534.

### e. Bromoacetyl-AOP-HP (Entry 5)

In a 0.5 mL Eppendorf tube, AOP-HP-2 (9.09 μL, 1.100 mmolar in DNase-free water, 10.0 nmol) was diluted with sodium borate (14.0 μL, 250.0 mmolar, pH=9.5, 3.50 μmol) and treated with 2,5-dioxopyrrolidin-1-yl 2-bromoacetate (3.00 μL, 200.0 mmolar in DMA, 600 nmol). The reaction was vortexed for 1 min and was left standing at 4° C. for 2 hours. The reaction mixture was precipitated (3.0 μL (3M NaOAc)+108 μL absolute ethanol). The crude DNA pellet was resuspended in DNase-free water (15.0 μL) and analyzed.

Bromoacetyl-AOP-HP-2 (10.40 nmol, 100%): OD: 1.040 mM; HPLC (gradient 4) Rₜ=5.40 min; MS(Orbitrap): calculated deconvoluted mass 17 241 found 17 241.

Bromoacetyl-AOP-HP-A-P1 (8.80 nmol, 88%): OD: 0.880 mM; HPLC (grad) Rₜ=5.06 min; MS(Orbitrap): calculated deconvoluted mass 12 156, found 12 156.

(iii) Headpiece Performance Comparison Under Various Deprotection and Diversification Reactions (Table 5)

### a. ((R)-azetidine-2-carbonyl)-AOP-HP (Entry 1)

N-Boc protected amino headpieces from Table 4, entry 1 were transferred to 200 μL PCR tubes, diluted with TRIS-HCl buffer (130 μL, 10 mM, pH=8.5, 1.30 μmol) and incubated at 85° C. for 16 h. Vials were allowed to cool down, transferred to fresh 1.5 mL Eppendorf tubes, and diluted with sodium borate buffer (24 μL, 250 mmolar, pH=9.4, 6.0 μmol). A modified precipitation procedure was employed due to the presence of TRIS: the crude DNA solutions were treated with sodium chloride (34 μL, 5 molar) and iPrOH (680 μL). Following precipitation, crude DNA pellets were resuspended in Dnase-free water (20.0 μL) and analyzed.

((R)-azetidine-2-carbonyl)-AOP-HP-2 (18.90 nmol, 95%): OD: 0.945 mM; HPLC (system 4) Rₜ=5.06 min; MS(TOF): calculated deconvoluted mass 17 204, found 17 204.

((R)-azetidine-2-carbonyl)-AOP-HP-A (18.63 nmol, 93%): OD: 0.932 mM; HPLC (system 4) Rₜ=4.72 min; MS(TOF): calculated deconvoluted mass 5 268, found 5 267.

### b. 4-piperidinecarbonyl-AOP-HP (Entry 2)

Nvoc-protected amino headpieces from step (ii-b) were diluted with sodium acetate buffer (30 μL, 50 mmolar, pH=4.5, 1.50 μmol) and placed in a reflective box kept at 4° C. The box was illuminated with blue LED, and the vials were incubated at 4° C. for 18 h, before they were precipitated (4.0 (3M NaOAc+0.3M MgCl₂)+160 μL absolute ethanol. The crude DNA pellets were resuspended in Dnase-free water (20.0 μL) and analyzed.

4-piperidinecarbonyl-AOP-HP-2 (1.84 nmol, 92%): OD: 0.092 mM; HPLC (system 2) Rₜ=6.99 min; MS(TOF): calculated deconvoluted mass 17 232, found 17 230.

4-piperidinecarbonyl-AOP-HP-A (1.36 nmol, 80%): OD: 0.068 mM; HPLC (system 2) Rₜ=5.18 min; MS(TOF): calculated deconvoluted mass 5 295, found 5 295.

### c. (2′-methyl-[1,1′-biphenyl])-4-carbonyl-AOP-HP (Entry 3)

In a 0.5 mL Eppendorf tube, 4-bromobenzoyl-AOP-HP-2 (3.30 μL, 0.908 mmolar, 3.0 nmol, from Table 4, entry 3) was diluted in sodium borate buffer (1.5 μL, 250 mmolar, pH=8.25, 0.38 μmol), treated with cesium hydroxide (1.25 μL, 600.0 mmolar in DNase-free water, 750 μmol) and phenylboronic acid (1.50 μL, 200.0 mmolar in Dioxane/DNase-free water (1:1), 300 nmol). The Eppendorf was vortexed for 1 minutes and treated with sSPhos-Pd-G2 (1.20 μL, 10 mM in DMA, 12 nmol). The Eppendorf was vortexed for 1 minute and left to stand at 80° C. for 30 minutes (centrifuged 10 sec at t=15 minutes). The Eppendorf was then centrifuged for 20 sec to ensure the liquid stay at the bottom of the Eppendorf. The reaction was treated with DTT (6.0 μL, 50 mM in Dnase-free water, 300 nmol), vortexed and left to stand 30 minutes at room temperature. The crude solution was diluted with Sodium Borate Buffer (6.000 μL, 250.0 mmolar, pH=9.5, 1.50 μmol), vortexed briefly and precipitated (2.1 μL (3M NaOAc)+100 μL absolute ethanol). The crude DNA pellet was resuspended in DNase-free water (20.0 μL) and analyzed.

(2′-methyl-[1,1′-biphenyl])-4-carbonyl-AOP-HP-2 (2.51 nmol, 82%): OD: 0.410 mM; HPLC (gradient 4) Rₜ=7.91; MS(TOF): calculated deconvoluted mass 17 314, found 17 314.

(2′-methyl-[1,1′-biphenyl])-4-carbonyl-AOP-HP-A-P1 (2.88 nmol, 96%): OD: 0.144 mM; HPLC (gradient 4) Rₜ=6.23; MS(Orbitrap): calculated deconvoluted mass 12 229, found 12 229.

### d. (1,1′-biphenyl)-4-carbonyl)-AOP-HP-T1 (Entry 4)

The protocol for entry 3 was repeated with headpiece AOP-HP-2 (3 nmol scale) as the electrophile and phenylboronic acid as the nucleophile. Precipitation yielded crude DNA pellets that were resuspended in Dnase-free water (15.0 μL) and analyzed.

[1,1′-biphenyl])-4-benzoyl-AOP-HP-2-T1 (2.81 nmol, 94%): OD: 0.140 mM; HPLC(HILIC) Rₜ=8.00; MS(TOF): calculated deconvoluted mass 22 871, found 22 871.

[1,1′-biphenyl])-4-benzoyl-AOP-HP-A-P1-T1 (2.88 nmol, 96%): OD: 0.144 mM; HPLC(HILIC) Rₜ=5.29; MS(Orbitrap): calculated deconvoluted mass 17 786, found 17 785.

### e. 4-((4-methoxyphenyl)amino)benzoyl-AOP-HP (Entry 5)

The protocol used for the synthesis of 4-(hydroxymethyl)benzoyl-AOP-HP was used with headpieces AOP-HP-2-T1 (30 μL, 1 mM in DNase-free water, 30 nmol) and AOP-HP-A-P1-T1 (30 μL, 1 mM in DNase-free water, 30 nmol) and precipitation followed by resuspension in Dnase-free water (30.0 μL) afforded crude 4-(hydroxymethyl)benzoyl-AOP-HP-2-T1 and 4-(hydroxymethyl)benzoyl-AOP-HP-A-P1-T1 which were used directly in the next step.

All solvent and water were degassed for 10 min by ultrasonication before use. In a 0.5 mL Eppendorf tube, 4-bromobenzoyl-AOP-HP-2-T1 (3.30 μL, 0.908 mmolar, 3.0 nmol) was treated with 4-methoxyaniline (3.0 μL, 500 mM in DMA, 1.50 μmol) and sodium hydroxide (2.10 μL, 1 molar in Dnase-free water, 2.10 μmol). The vials were vortexed for 30 seconds and degassed by ultrasonication for 2 min and to enhance mixing. Then, allylpalladium(II) Chloride (0.18 μL, 10 mmolar in DMA, 1.80 μmol), cBRIDP (450.0 nL, 10 mmolar in DMA, 4.50 nmol) and DMA (0.9 μL) were added to yield a solution of roughly 1:1 DMA/H₂O final ratio. The reaction was vortexed for 10 second and degassed by ultrasonication for 2 min. The sample was then capped and heated at 80° C. for 1 h using a dry block heater. The Eppendorf was centrifuged for 20 sec to ensure the liquid stay at the bottom of the Eppendorf. The reaction was treated with DTT (6.0 μL, 50 mM in DNase-free water, 300 nmol), vortexed and left to stand 30 minutes at room temperature. The crude solution was diluted with Sodium Borate Buffer (6.000 μL, 250 mmolar, pH=9.5, 1.50 μmol), vortexed briefly and precipitated (2.1 μL (3M NaOAc)+100 μL absolute ethanol). The crude DNA pellet was resuspended in Dnase-free water (25.0 μL) and analyzed.

A closing primer was ligated as described previously and the headpiece was purified by preparative HPLC. Collected fractions were evaporated, resuspended in Dnase-free water (20.0 μL) and analyzed.

4-((4-methoxyphenyl)amino)benzoyl-AOP-HP-2-T1 (1.27 nmol, 42%): OD: 0.063 mM; HPLC(HILIC) Rₜ=7.40 min; MS(TOF): calculated deconvoluted mass 22 916, found 22 916.

4-((4-methoxyphenyl)amino)benzoyl-AOP-HP-A-P1-T1 (1.24 nmol, 41%): OD: 0.062 mM; Rₜ=6.78 min; MS(TOF): calculated deconvoluted mass 17 831, found 17 830.

4-((4-methoxyphenyl)amino)benzoyl-AOP-HP-AOP-HP-A-P1-T1-T2-CP (0.12±0.05 nmol, 12±5%): OD: 0.008±0.002 mM; HPLC (gradient 2) Rₜ=7.68 min; Gel: Agarose, 75 bp.

3-(5-(3-hydroxypropyl)-1H-1,2,3-triazol-4-yl)benzoyl-AOP-HP-A-P1-T1-T2-CP (0.53±0.14 nmol, 53±14%): OD: 0.013±0.004 mM; HPLC (gradient 2) Rₜ=6.47 min; Gel: Agarose, 75 bp.

### f. 3-(5-(3-hydroxypropyl)-1H-1,2,3-triazol-4-yl)benzoyl-AOP-HP (Entry 6)

The protocol used for the synthesis of 4-(hydroxymethyl)benzoyl-AOP-HP was used with headpieces AOP-HP-2-T1 (15 μL, 1 mM in DNase-free water, 15 nmol) and AOP-HP-A-P1-T1 (15 μL, 1 mM in DNase-free water, 15 nmol) and precipitation followed by resuspension in Dnase-free water (15.0 μL) afforded crude 3-ethynylbenzoyl-AOP-HP-2-T1 and 3-ethynylbenzoyl-AOP-HP-A-P1-T1 which were used directly in the next step.

In a 0.5 mL Eppendorf tube, 3-ethynylbenzoyl-AOP-HP-2-T1 (3.94 μL, 0.507 mmolar in Dnase-free water, 2.0 nmol) was treated with 2-azidoethan-1-ol (1.00 μL, 200 mmolar in DMA, 200 nmol), followed by a pre-mixed solution of copper sulfate (0.40 μL, 50 mmolar in Dnase-free water, 20.0 nmol), THPTA (0.60 μL, 200.0 mmolar in DNase-free water, 120 nmol) and sodium ascorbate (0.80 μL, 50 mmolar, 20 Eq, 0.0400 μmol). The reaction was left to stand 1 h at room temperature. The reaction was treated with DTT (4.00 μL, 50 mmolar in DNase-free water, 200 nmol), vortexed and left to stand 30 minutes at room temperature. The crude solution was precipitated (1.3 μL (3M NaOAc)+50 μL absolute ethanol) and the crude DNA pellet was resuspended in DNase-free water (20.0 μL) and analyzed.

A closing primer was ligated as described previously and the headpiece was purified by preparative HPLC. Collected fractions were evaporated, resuspended in Dnase-free water (20.0 μL) and analyzed.

3-(5-(3-hydroxypropyl)-1H-1,2,3-triazol-4-yl)benzoyl-AOP-HP-2-T1 (3.61 nmol, 100%): OD: 0.181 mM; HPLC(HILIC) Rₜ=7.24 min; MS(TOF): calculated deconvoluted mass 22 906, found 22 906.

3-(5-(3-hydroxypropyl)-1H-1,2,3-triazol-4-yl)benzoyl-AOP-HP-A-P1-T1 (2.12 nmol, 100%): OD: 0.106 mM; HPLC(HILIC) Rₜ=6.88 min; MS(TOF): calculated deconvoluted mass 17 821, found 17 820.

4-((4-methoxyphenyl)amino)benzoyl-AOP-HP-AOP-HP-2-T1-T2-CP (0.27±0.04 nmol, 27±4%): OD: 0.045±0.003 mM; HPLC (gradient 2) Rₜ=5.97 min; Gel: Agarose, 80 bp.

3-(5-(3-hydroxypropyl)-1H-1,2,3-triazol-4-yl)benzoyl-AOP-HP-2-T1-T2-CP (0.51±0.08 nmol, 51±8%): OD: 0.013±0.002 mM; HPLC (gradient 2) Rₜ=7.35 min; Gel: Agarose, 80 bp.

### Example 7—Conversion of HP-1/HP-2 in ssdNA DELs

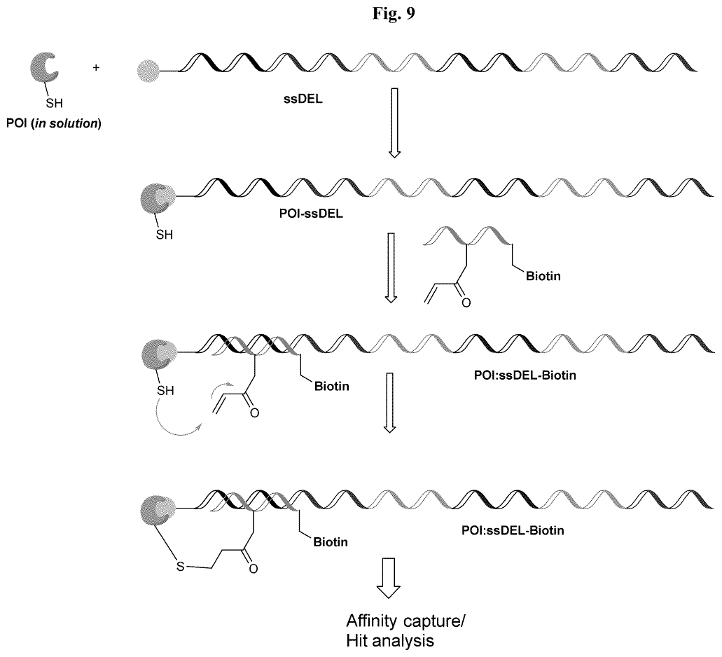

Having the ability to interconvert between single- and double-stranded headpieces represent an advantage for certain types of screening modalities, such as when one, or multiples, secondary molecular effectors are required (see “Example 8” below). Examples of secondary molecular effectors use to access advanced DEL screening modalities includes in-solution selections, screens in live cells or complex biological media, or directed screens using modified proteins of interest, are well described for ssDEL (FIG. 9), where annealing of a short, functionalized oligonucleotides complementary to the single stranded sequence provide an ideal pathway to add different effectors in function of the screen to be performed (Huang et al.). Examples of these advanced screening modalities in dsDEL are scarce, mostly because of the lack of suitable ssDNA region to base-pair functionalized DNA with. Since two copies of the pharmacophore identity are present in dsDEL, complete or partial removal of the lower strand by enzymatic degradation is not expected to influence library amplifiability significantly, and represent an attractive way of combining the advantages of dsDEL (chemical resistance, ease of ligation) with the high screening versatility offered by ssDEL.

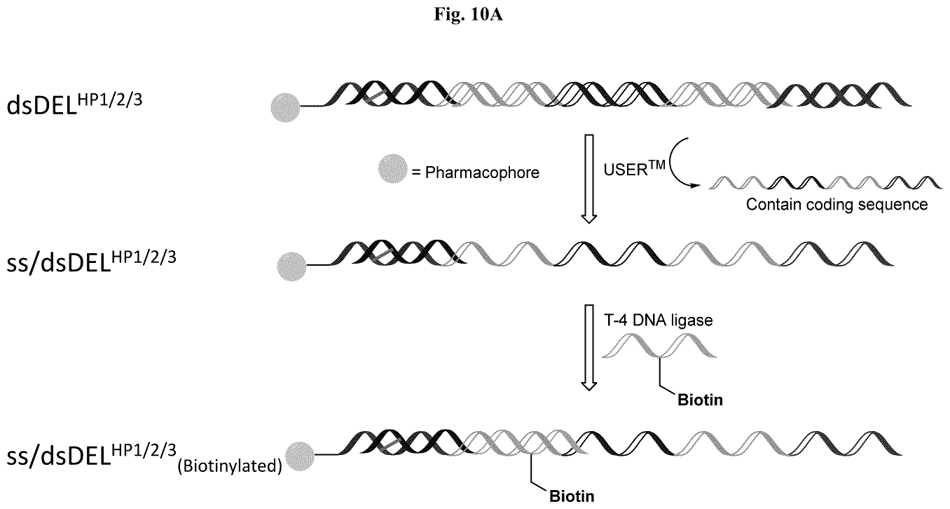

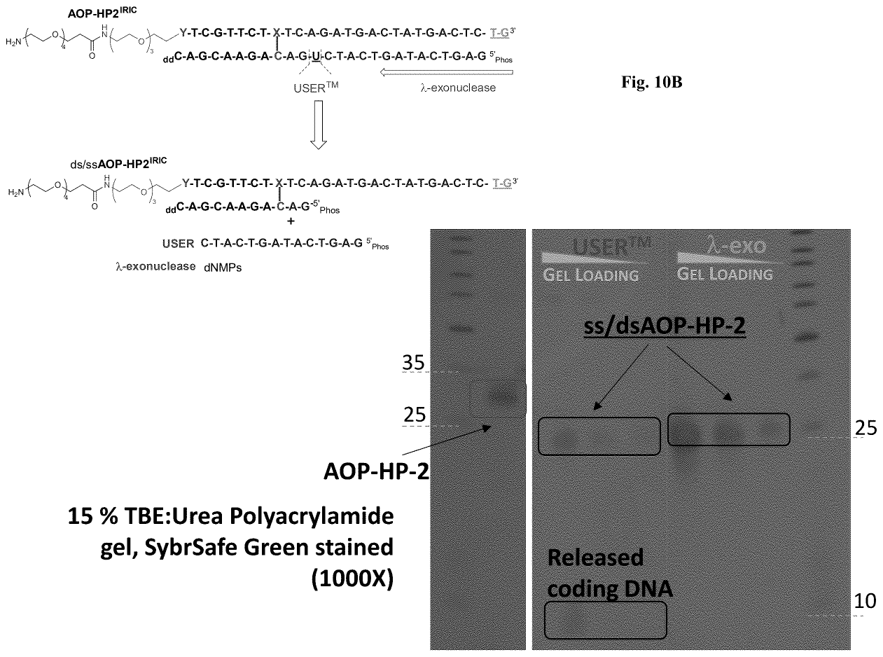

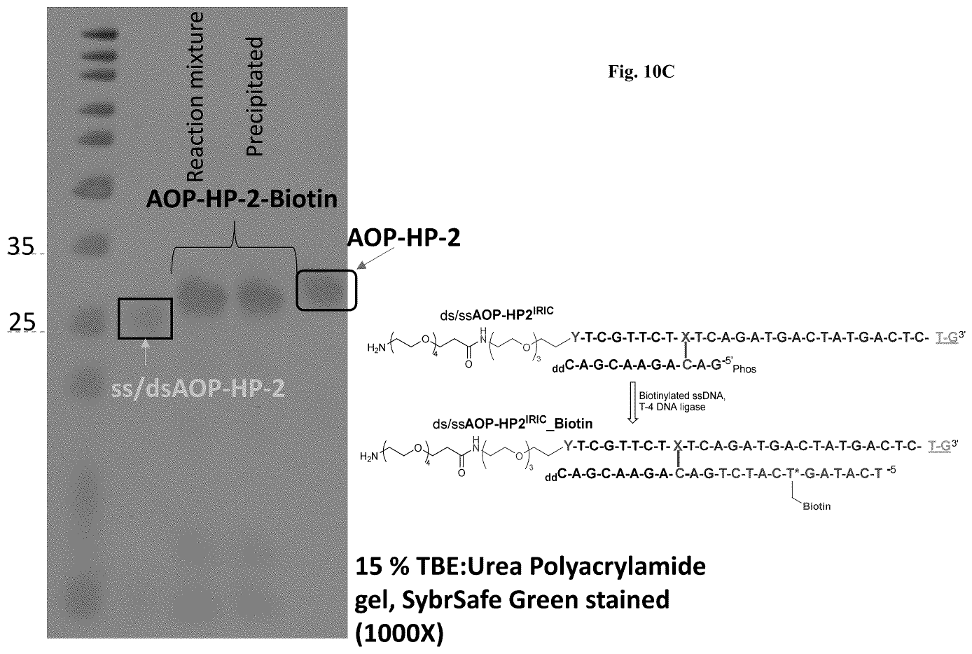

HP-1/2/3 sequence contains either one or two deoxy-uracil residues located 5′ to the reading frame, and said residue is susceptible to enzymatic excision followed by single strand break by USER™ enzymes mix, providing an elegant way to release one of the two coding DNA strands. Moreover, since USER (USER (Uracil-Specific Excision Reagent; Uracil DNA glycosylase+Endonuclease Vill)) leaves a phosphate on the 5′-termini at the site of excision, ligation of small ssDNA complementary to the sense strand can be done via T-4 DNA ligase. This enables the addition of secondary molecular effectors to the pharmacophore-DNA construct in a fashion similar to some ssDEL protocols, but with the advantage of covalent attachment (FIG. 10A). Example of secondary molecular effectors includes, without being limited to, affinity capture-tags (ex.: Biotin, His-Tag) and groups designed to covalently crosslink the protein of interest (ex.: Michael acceptors, Methyl azetidines, Benzophenones). If multiple dU residues are present, such as in AOP-HP-3, USER™ III enzyme mix is then used, leaving behind a 5′-phosphate group than can then serve as ssDNA anchoring point, while removing the 3′-phosphate to allow unhindered non-covalent hybridization with a functionalized ssDNA. This last functionalization is reversible, as the DNA strand is not linked trough the backbone and will be released upon heating.

To demonstrate the DNA release and partial conversion to ssDEL, HP-2 was treated with USER™ enzyme and analyzed by denaturing PAGE (FIG. 10B and FIG. 10C). Results clearly indicate that the cleavage occurred at the desired position, and with release of intact ssDNA, which, in the context of a DEL library would contain the pharmacophore identity code. A-exonuclease was also found to perform antisense strand degradation well, however, while the reaction yields ss/dsAOP-HP-2 as the major product, traces of 5′-guanosine removal were also present. Additionally, this enzymatic reaction degrades the antisense strand completely, destroying the information contained, and requires a 5′-Phosphate modification to work. In light of these results, USER™ enzyme mix was selected for further development. To demonstrate that a functionalized oligonucleotide can be ligated, a short Biotinylated oligonucleotide was purchased and ligated to ss/dsAOP-HP-2 using standard T-4 enzyme protocols, yielding ss′/ds′_{(biotin)}AOP-HP-2 which was analyzed by denaturing PAGE (FIG. 10C) to confirm covalent ligation.

ss/dsAOP-HP-2 Preparation (USER™)

In a 0.5 mL microcentrifuge tube, AOP-HP-2 (0.60 μL, 0.899 mM in water, 0.54 nmol) was diluted with Dnase-free water (400 μL) and rCutSmart Buffer™ (10×) (50.00 μL), mixed on the vortexer, and treated with USER™ Enzyme (1000 U/mL) (25.00 μL, 25 U). The mixture was well mixed with the vortexer and incubated at 37° C. for 1 h before being allowed to cool down to room temperature. The vial was left to stand for ca 18 h, after which a sample (5 μL) was collected for PAGE analysis and DNA was precipitated. The crude product was dissolved in Dnase-free water (20.0 μL) and analyzed by denaturing PAGE.

ss/dsAOP-HP-2 Preparation (λ-Exonuclease)

In a 0.5 mL microcentrifuge tube, AOP-HP-2_IRIC (1.10 μL, 0.899 mmolar in water, 0.99 nmol) was diluted with Dnase-free water (55.00 μL, 1 Eq, 0.99 nmol) and treated with Lambda Exonuclease Buffer (10×) (7.00 μL) and Lambda Exonuclease (5000 U/mL) (7.00 μL, 35 U). The vial was vortexed and incubated for 1 h at 37° C., before being allowed to cool down to room temperature left to stand for ca 18 h. The vial was treated with aqueous EDTA (7.00 μL, 100.0 mmolar in water, 0.70 μmol) and the enzyme was heat-inactivated at 75° C. for 10 min. A sample was collected for Page analysis and the vial was allowed to cool down to RT and centrifuged to pellet insoluble. The supernatant was transferred to a fresh tube and DNA was precipitated. The crude DNA pellet was dissolved in Dnase-free water (20.0 μL) and analyzed by denaturing PAGE.

ss′/ds′_{(biotin)}AOP-HP-2

In a 0.5 mL microcentrifuge tube, 10 μL of the crude USER-mediated ss/dsAOP-HP-2 conversion reaction was treated with biotinylated ssDNA (TCATAG(T[Biotin])CATCT [SEQ ID NO: 10]). Dnase-free water was added, followed by 10× ligation buffer and T-4 enzyme, as described above. The reaction mixture was incubated at 30° C. for ca 18 h, at which point 2 μL of the reaction mixture were sampled and mixed with TBU loading buffer containing SDS for PAGE analysis. The reaction was precipitated, DNA was resuspended in Dnase-free water (20.0 μL) and both the reaction mixture and precipitated products were analyzed by denaturing PAGE (FIG. 10c).

### Example 8—HP-1/2/3-Enabled Covalent-DEL Multidimensional Screens

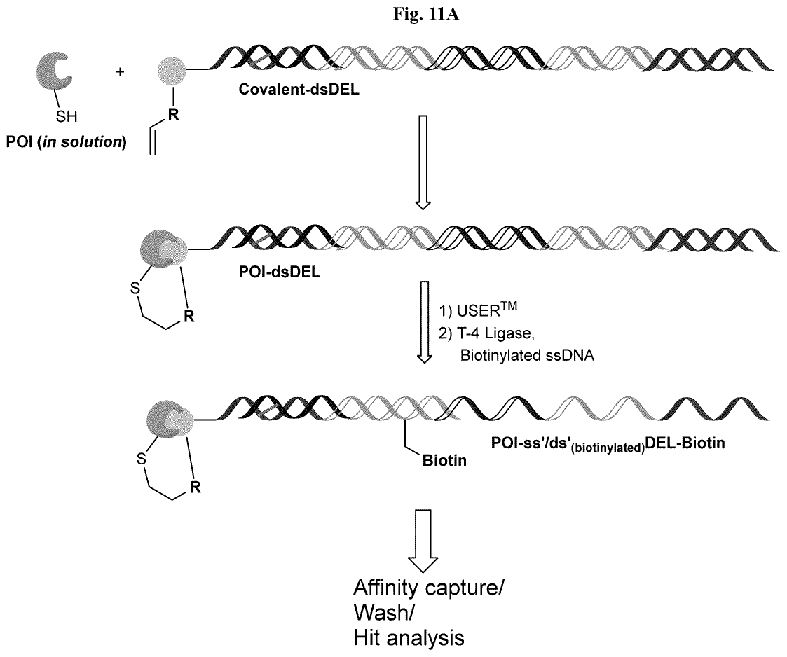

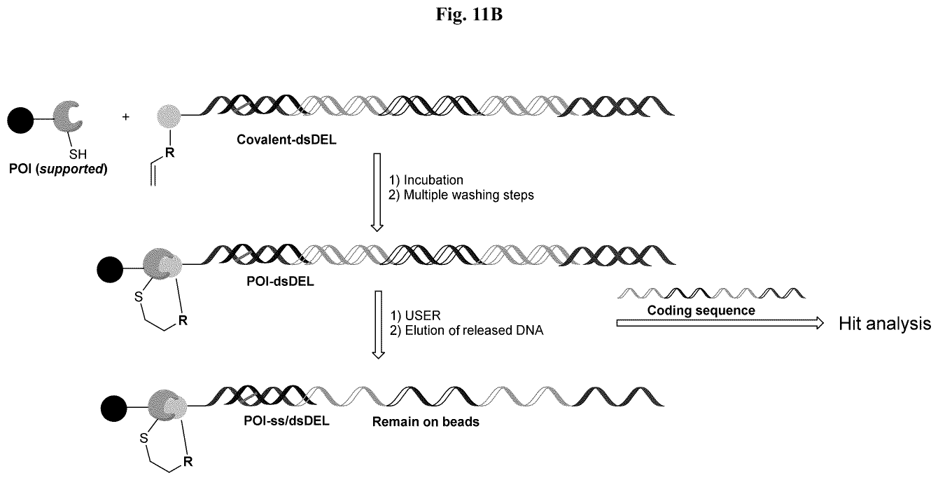

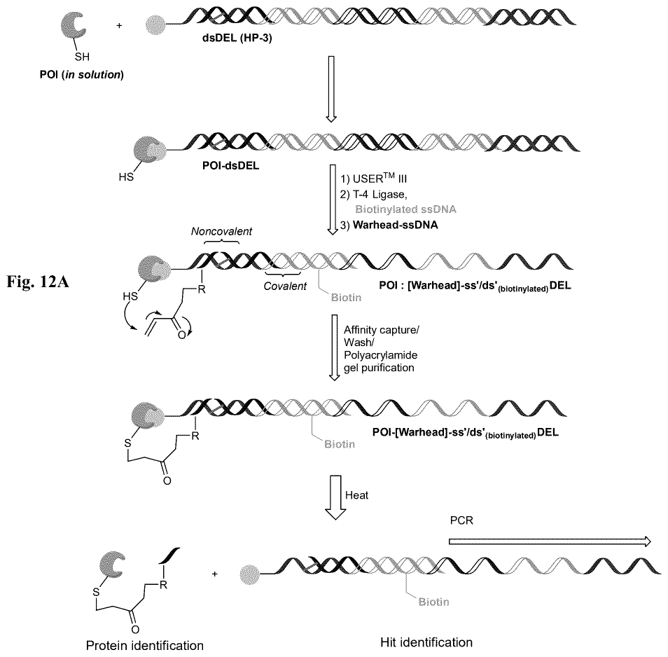

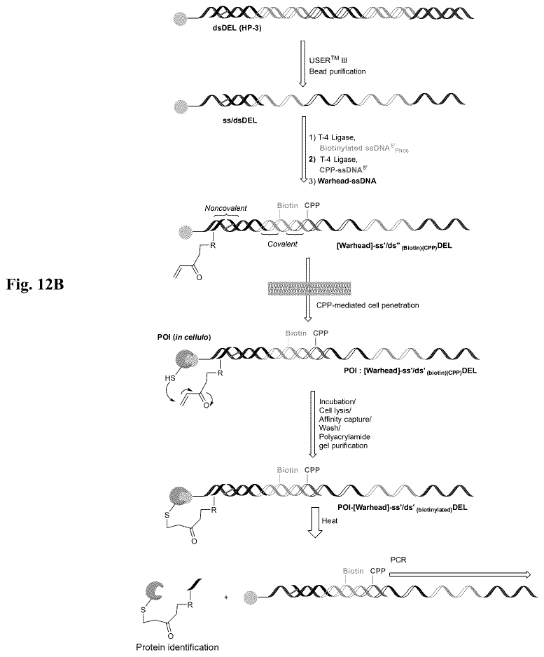

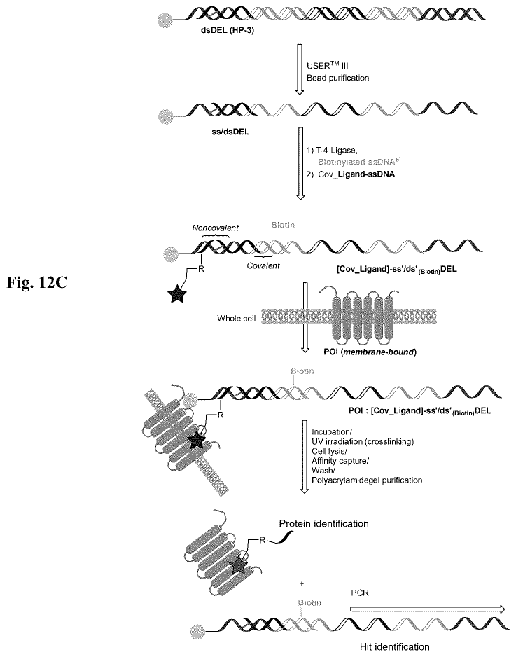

A schematic representation of applications where the conversion between ssAOP-HP-1/2/3 and dsAOP-HP-1/2/3 may be advantageous to improve screening efficiency can be seen in FIGS. 11A-11B, and FIG. 12A-12D. In the case of a library of pharmacophores designed to bind covalently to their target trough reactive moieties, ss′/ds′_{(biotin)} AOP-HP-2 would enable the development of solution-phase screening protocols. Briefly, the library and targets are incubated together, and dsAOP-HP-2 is converted to ss′/ds′_{(biotin)} AOP-HP-2. Following ligation of a biotinylated short single strand sequence to the remaining headpiece, the POI-ss′/ds′DEL-Biotin complexes can be washed to remove any nonbinders and sequenced to reveal hit identity (FIG. 11A). The same library can also be conveniently screened against a solid-supported target, by incubating both beads and library together, performing standard washing steps, and releasing the single-strand coding sequence enzymatically (FIG. 11B). Protocols presented offer significant advantages over conventional dsDEL, as they enable screening of covalent libraries against targets that are not amenable to solid immobilization and give the opportunity to perform the DEL selection on proteins in much more biologically relevant solution state.

Since ssAOP-HP-3 also offers the possibility to add secondary molecular effectors both covalently and non-covalently, more complex screening modalities taking advantage of both reversible and non-reversible functionalization, such as in-solution screens against unmodified proteins of interest (FIG. 12A), in cellulo and (FIG. 12B) membrane bound screens (FIG. 12C) can be envisioned. In those cases, HP-3 is functionalized with a covalent warhead, which may or may not possess intrinsic affinity for the POI, in a noncovalent fashion, and with a biotin affinity capture tag in a covalent manner. In addition, if the screening is to be performed inside living cells, a cell-penetrating peptide moiety is added covalently. Following incubation with the target, a crosslinking step is performed to irreversibly link the covalent warhead to the POI. Following cell lysis or membrane denaturation, the POI-DEL complexes are separated using streptavidin beads or columns, and purified by non-denaturating PAGE. Once the complexes are isolated, the warhead-POI moiety is released by heating, DNA is amplified and sequenced to reveal the hit's identity and the POI bearing the covalent warhead is characterized by standard protein characterization methodologies. The combination of reversible and non-reversible molecular effectors can also be used to develop cooperative binding assays in which a PPI-biased DEL can be functionalized reversibly with a secondary protein trough complementary base-pairing of two short ssDNA tags, and irreversibly with a photo-crosslinker and an affinity capture tag (FIG. 12D). Using this strategy, a secondary complex between the secondary POI and the library can be formed before addition of the primary POI to the solution. The tertiary complexes are then pulled from the selection matrix using streptavidin beads and washed with buffer, irradiated to achieve photo-crosslinking, and heated to remove the reversible secondary protein effector. Binders crosslinked to the protein of interest are then isolated from non-crosslinked DEL by non-denaturing PAGE, amplified and sequenced to afford hit's identity.

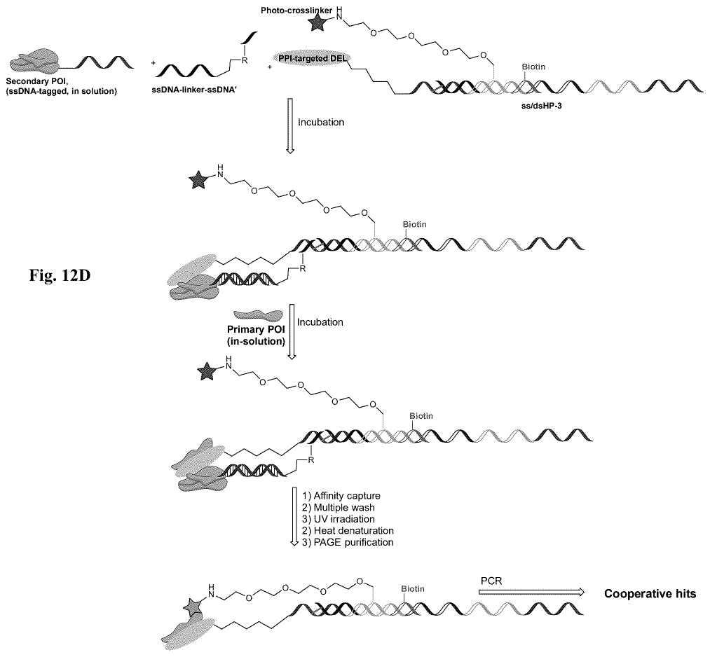

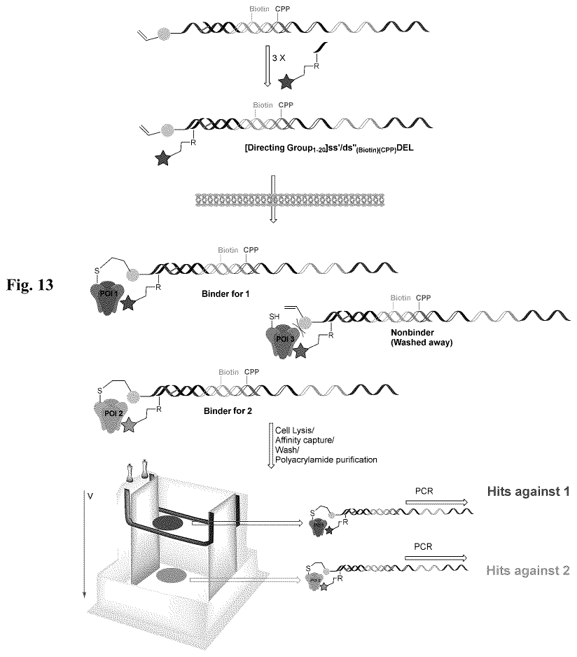

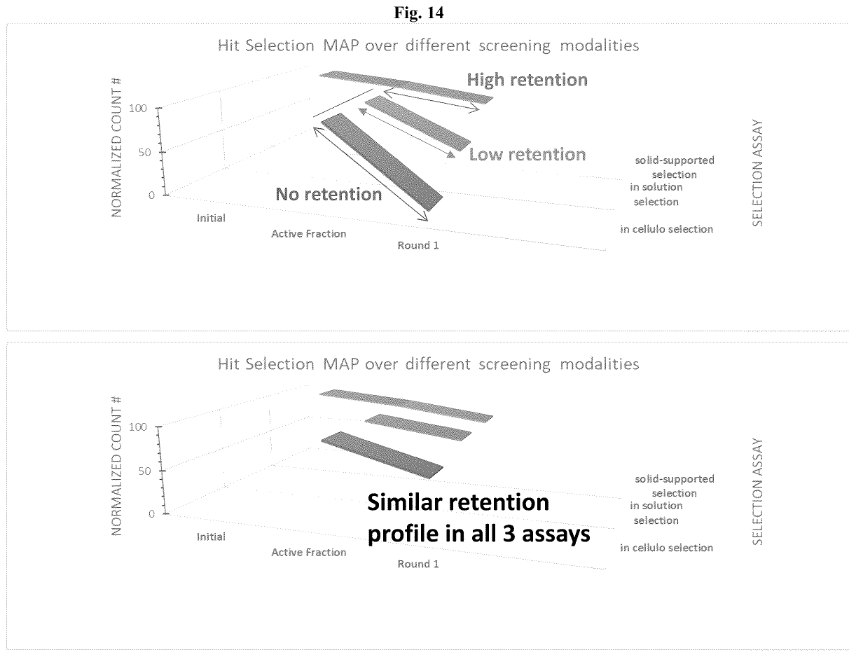

Using this strategy in conjunction with covalent DEL, multiple POI can be screened in the same biological matrix by varying the nature of the warhead in a dynamic fashion, allowing to target complete biological pathways rather than distinct proteins, so long that they can be purified from one another by PAGE and identified (FIG. 13). While advanced screening modalities definitely expand the scope of possible targets to be studied by DEL, they also offer the opportunity to generate considerably more information about the behavior of each hit composing the library. FIG. 14 illustrate the advantage of being able to perform screens in more than one modality to allow multidimensional analysis. While the compound depicted at the top has favorable profile when selection is performed a solid supported setting, poor performances in the in-solution and in vivo screens may be indicative that the hit isn't active against the protein when it is free to adopt its native conformation or in more complex biological matrices. In the case of the compound depicted at the bottom, however, the retention profile is conserved across screening modalities, potentially indicating a much more relevant hit. Multidimensional (more than one screening modality) DEL analysis may be more time and resources consuming, but they have the potential to drastically diminish false negative rates, and the large datasets of results they generate can be processed using standard bioinformatic tools or using artificial intelligence algorithms.

### Example 9—Discussion

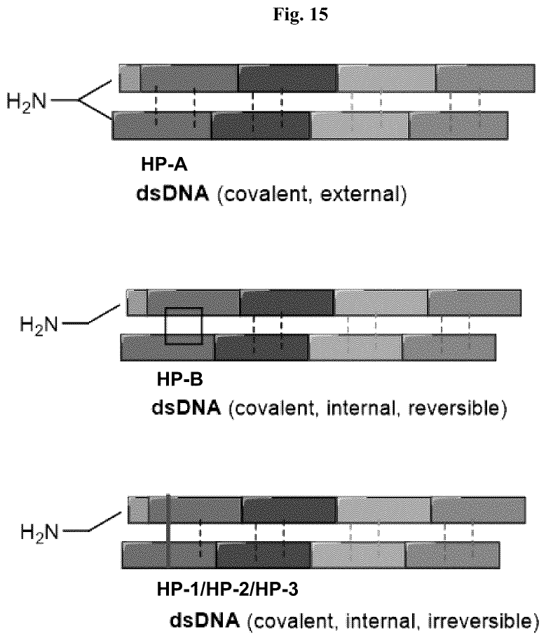

In summary, we have developed a novel headpiece technology for the development of DNA-encoded libraries. The present design relies on the use of inter-strands, irreversible covalent cross-linking, which was shown to help expand the available chemical space by imparting greater tolerance to chemical conditions relative to available alternatives such as those described in the HP described in WO2005058479A2 (HP-A) and Zhao et al. (HP-B) (FIGS. 10A-10C and FIG. 15). The headpiece developed has been shown to perform enzymatic ligation more efficiently than the conventional HP-A, and also resulted in improve ethanol precipitations yields and optical density measurements precision. In addition, HP-1/2/3 was shown to perform better than HP-A under multiple reaction conditions with respect to DNA recovery after precipitations. The increased chemical resistance of HP-1/2/3 was explored under a variety of highly strenuous conditions, revealing improved resistance against low pH, Metal catalysts (Pd, Cu) and Cerium triflate. The ability to release coding DNA through a restriction enzyme may also prove useful for certain types of screens, most specifically screens of covalent DEL. Finally, has been shown that HP-1/HP-2 can be partially converted to ssDNA through the action of either USER™ I-Ill enzymes mix or A-exonuclease, paving the way for more complex screening modalities, such as screening for POI directly in solution or multimodal screening modalities (FIGS. 11A-11B, and FIGS. 12A-12D).

Numerous modifications could be made to any of the embodiments described above without departing from the scope of the present invention. Any references, patents or scientific literature documents referred to in the present document are incorporated herein by reference in their entirety for all purposes.

### REFERENCES

Gui, Y.; Wong, C. S.; Zhao, G.; Xie, C.; Hou, R.; Li, Y.; Li, G.; Li, X., Converting Double-Stranded DNA-Encoded Libraries (DELs) to Single-Stranded Libraries for More Versatile Selections. ACS Omega 2022.
WO2005058479A2
WO2018166532A1
Zhao, G.; Zhong, S.; Zhang, G.; Li, Y.; Li, Y., Reversible Covalent Headpiece Enables Interconversion between Double- and Single-Stranded DNA-Encoded Chemical Libraries. Angewandte Chemie 2022, 134 (7), e202115157.
Huang Y, Li Y, Li X., Strategies for developing DNA-encoded libraries beyond binding assays Nature chemistry. 2022; 14(2):129-40.
Shi, B.; Deng, Y.; Zhao, P.; Li, X., Selecting a DNA-Encoded Chemical Library against Non-immobilized Proteins Using a “Ligate-Cross-Link-Purify” Strategy. Bioconjugate Chemistry, 2017; 28: 2293-2301.

1. A headpiece, wherein said headpiece comprises a first polynucleotide sequence and a second polynucleotide sequence, wherein:
said second polynucleotide sequence comprises a sequence that is complementary to the first polynucleotide sequence;
at least part of said first and second polynucleotide sequences forms a double stranded polynucleotide sequence;
said first and second polynucleotide sequences are covalently and irreversibly linked to each other through at least one modified base pair present in the double stranded polynucleotide sequence; and
said first and second polynucleotide sequences each comprise a first and a second end, wherein:
one of the first ends comprises a functionalizable moiety attached through a linker;
the other of the first ends comprises a chain termination nucleotide; and
the second ends comprise a DNA anchor site.

2. A headpiece, wherein said headpiece comprises a first polynucleotide sequence (e.g., sense strand) and a second polynucleotide sequence (e.g., antisense strand), wherein:
said second polynucleotide sequence comprises a sequence that is complementary to the first polynucleotide sequence;
at least part of said first and second polynucleotide sequences forms a double stranded polynucleotide sequence;
said first and second polynucleotide sequences are covalently and irreversibly linked to each other through at least one modified base pair present in the double stranded polynucleotide sequence; and
said first and second polynucleotide sequences each comprise a 5′- and a 3′-end, wherein:
the 5′ end of the first polynucleotide sequence comprises a functionalizable moiety attached through a linker;
the 5′ end of the second polynucleotide sequence comprises a chain termination nucleoside, preferably a dideoxynucleoside;
the second polynucleotide sequence comprises one or more deoxy-uridine residues used as enzymatic cleavage sites; and
the 3′ end of the first polynucleotide sequence comprises a two-nucleotide single strand overhang and the 5′ ends of the second polynucleotide sequence comprise a 5′-phosphate, thereby creating a DNA anchor site.

3. The headpiece of claim 1 or 2, wherein said at least one modified base pair comprises a crosslink selected from an alkyl, alkylamine, or alkoxy group comprising a 3-atom chain.

4. The headpiece of any one of claims 1 to 3, wherein said modified base pair is of the formula: 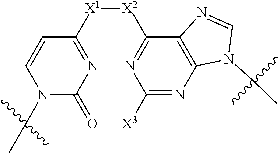
wherein:
X¹ and X² taken together form a —CR₂—CR₂—CR₂—, —CR₂—CR₂—NH—, —NH—CR₂—CR₂—, —CR₂—CR₂—O—, or —O—CR₂—CR₂— group, wherein R is independently H or methyl, preferably R is H in all instances;
X³ is selected from H and NH₂;
each of the base being attached to a phosphorylated sugar opposite to each other in the first and second polynucleotide sequences.

5. The headpiece of claim 4, wherein X¹ and X² taken together form a —NH—CR₂—CR₂— group, preferably a —NH—CH₂—CH₂— group.

6. The headpiece of any one of claims 1 to 5, wherein said modified base pair is located between the 4th and 15th base from the linker of the functionalizable moiety, preferably between the 6th and 10th base

7. The headpiece of any one of claims 1 to 6, wherein said double stranded polynucleotide sequence comprises from 15 to 35 base pairs, preferably 20 to 30 base pairs.

8. The headpiece of any one of claims 1 to 7, wherein said first polynucleotide sequence comprises the functionalizable moiety attached through the linker.

9. The headpiece of claim 8, wherein the first end of the second polynucleotide sequence comprises the chain termination nucleotide.

10. The headpiece of any one of claims 1 to 9, wherein the chain termination nucleotide is a dideoxynucleoside.

11. The headpiece of claim 10, wherein the chain termination nucleotide is a dideoxycytidine.

12. The headpiece of any one of claims 1 to 11, wherein the linker from the functionalizable moiety comprises a polyether chain, preferably a di(ethylene glycol), tri(ethylene glycol) or tetra(ethylene glycol) chain, attached to the phosphorylated nucleotide present at the first end of the polynucleotide sequence.

13. The headpiece of any one of claims 1 to 12, wherein the functionalizable moiety comprises an amine group, preferably a primary amine group, an azide group or a functional group comprising a reactive moiety (e.g., an iodo-phenyl or bromo-phenyl group).

14. The headpiece of claim 13, wherein the functionalizable moiety comprises a primary amine group, for example the linker being a polyether, preferably the polyether being attached through a deoxy-nucleotide, e.g., the functionalizable moiety and linker together being a 5′-amino-PEG-deoxy-nucleotide, wherein PEG comprises between 2 and 5 ethylene glycol units (such as 5′-amino-PEG3-deoxy-guanosine).

15. The headpiece of any one of claims 1 to 14, wherein the functionalizable moiety serves as an attachment point for a pharmacophore to be tested in a DNA-encoded library.

16. The headpiece of any one of claims 1 to 15, wherein said double stranded polynucleotide sequence further comprises one or more cleavage sites.

17. The headpiece of claim 16, wherein said cleavage sites comprise one or more uridines (e.g., deoxy-uridines) in one of the first and second polynucleotide sequences, preferably in the second polynucleotide sequence.

18. The headpiece of any one of claims 1 to 17, further comprising an integrated primer sequence.

19. The headpiece of claim 18, wherein the primer sequence comprises a forward strand that is at least 70%, 80%, 90%, 95%, or 100% identical to SEQ ID NO: 3, and/or a reverse strand that is at least 70%, 80%, 90%, 95%, or 100% identical to SEQ ID NO: 4.

20. The headpiece of any one of claims 1 to 19, comprising an antisense strand that is at least 70%, 80%, 90%, 95%, or 100% identical to the sequence of SEQ ID NO: 1, 5, or 7; and/or a sense strand that is at least 70%, 80%, 90%, 95%, or 100% identical to the sequence of any one of SEQ ID NOs: 2, 6, or 8.

21. The headpiece of any one of claims 1 to 20, wherein said first and/or second polynucleotide sequences comprise nucleoside analogs, such as locked nucleic acid residues, 2′-alkylated RNA residues, peptide nucleic acid residues, and/or 2′-fluoro DNA residues.

22. The headpiece of any one of claims 1 to 21, wherein said first and/or second polynucleotide sequences comprise phosphodiester or phosphonothioate linkages.

23. The headpiece of any one of claims 1 to 21, wherein said first and/or second polynucleotide sequences comprise an affinity-pairing region for binding to one or more secondary molecular effectors.

24. The headpiece of claim 23, wherein said binding is reversible.

25. The headpiece of claim 23 or 24, wherein said secondary molecular effectors comprise biotin groups, photoreactive groups, cell-penetrating peptides, secondary protein recruiters, and/or fluorophores.

26. The headpiece of any one of claims 1 to 25, for use in a DNA-encoded library (DEL) (e.g., double-stranded or single-stranded DEL).

27. The headpiece as defined in any one of claims 1 to 25, for use in anchoring a control molecule, preferably for use in a pulldown biological assay.

28. A DNA-supported pharmacophore comprising a headpiece as defined in any one of claims 1 to 25, and a pharmacophore attached to the functionalizable moiety of the headpiece.

29. The DNA-supported pharmacophore of claim 28, further comprising a primer attached to the DNA anchor site of the headpiece.

30. The DNA-supported pharmacophore of claim 29, wherein the primer comprises a forward strand that is at least 70%, 80%, 90%, 95%, or 100% identical to SEQ ID NO: 3, and/or a reverse strand that is at least 70%, 80%, 90%, 95%, or 100% identical to SEQ ID NO: 4.

31. The DNA-supported pharmacophore of any one of claims 28 to 30, further comprising at least one DNA tag sequence attached to the primer.

32. The DNA-supported pharmacophore of claim 31, comprising between 2 and 5 DNA tag sequences, preferably 3 to 5 DNA tag sequences, more preferably 4 DNA tag sequences, wherein when said DNA tag sequences are sequentially ligated.

33. The DNA-supported pharmacophore of claim 31 or 32, wherein said DNA tag sequence(s) each comprise between 5 and 12 base pairs, preferably between 6 and 10 base pairs.

34. The DNA-supported pharmacophore of any one of claims 28 to 33, further comprising a closing primer attached to the free end of the last DNA tag sequence.

35. A DNA-encoded library (DEL) (e.g., double-stranded or single-stranded DEL) comprising a plurality of DNA-supported pharmacophores as defined in any one of claims 28 to 34.

36. Use of the headpiece as defined in any one of claims 1 to 25, for the preparation of a DNA-encoded library (DEL) (e.g., double-stranded or single-stranded DEL).

37. Use of the headpiece as defined in any one of claims 1 to 25, for anchoring a control molecule, preferably for use in a pulldown biological assay.

38. A method for the preparation of a headpiece as defined in any one of claims 1 to 25, the method comprising:
preparing a 6-vinyl purine precursor;
preparing the first polynucleotide sequence comprising the 6-vinyl purine precursor and converting the 6-vinyl purine precursor into a 6-vinyl purine (e.g., sense strand);
preparing the second polynucleotide sequence (e.g., antisense strand); and
contacting the first polynucleotide sequence and second polynucleotide sequence to form a covalently linked double stranded (HP) wherein the vinyl group of the 6-vinyl purine reacts with an amine group of a base facing the 6-vinyl purine on the second sequence.

39. A method for preparing a DNA-encoded library (DEL) (e.g., double-stranded or single-stranded DEL), said method comprising:
preparing the headpiece as defined in any one of claims 1 to 25, optionally wherein the headpiece comprises an integrated primer;
contacting the headpiece with a pharmacophore comprising a functional group reactive to the functionalizable moiety to covalently link the pharmacophore with the functionalizable moiety;
optionally, attaching a primer to the DNA anchor site of the headpiece; and
optionally, attaching at least one DNA tag sequence to the primer.

40. The method of claim 39, wherein the attached or integrated primer comprises a forward strand that is at least 70%, 80%, 90%, 95%, or 100% identical to SEQ ID NO: 3, and/or a reverse strand that is at least 70%, 80%, 90%, 95%, or 100% identical to SEQ ID NO: 4.

41. The method of claim 39 or 40, wherein step (d) comprises attaching between 2 and 5 DNA tag sequences, preferably 3 to 5 DNA tag sequences, more preferably 4 DNA tag sequences, wherein when said DNA tag sequences are sequentially ligated.

42. The method of any one of claims 39 to 41, wherein said DNA tag sequence(s) each comprise between 5 and 12 base pairs, preferably between 6 and 10 base pairs.

43. The method of any one of claims 39 to 42, further comprising attaching a closing primer to the free end of the last DNA tag sequence.

44. A method for adding secondary molecular effectors to a DNA-encoded library (DEL) (e.g., double-stranded or single-stranded DEL), said method comprising:
a) leaving the second polynucleotide sequence of the headpiece as defined in any one of claims 1 to 25 at the enzymatic cleavage sites by treatment with a uracil-specific excision reagent (e.g. USER™ I, II or III, preferably USER™ I or III);
b) introducing a first molecular effector by hybridizing a first functionalized single-stranded oligonucleotide of complementary sequence 5′ of the crosslinking base-pair, and ligating with T-4 DNA ligase;
c) repeating step (b) as required until the length of the functionalized second polynucleotide sequence remains shorter that that of the first polynucleotide sequence of the headpiece as defined in any one of claims 1 to 25; and
d) optionally, adding one last, non-covalently bound, secondary molecular effector by hybridizing a functionalized single-stranded oligonucleotide of complementary sequence 3′ of the crosslinking base-pair.

45. The method of claim 44, wherein the secondary molecular effectors comprise biotin groups, photoreactive groups, cell-penetrating peptides, secondary protein recruiters, and/or fluorophores.

46. The method of any one of claims 39 to 45, wherein the DEL is the DEL as defined in claim 35.


SEQUENCE LISTING (10 sequences)

SEQ ID NO: 1 - DNA, 26 bp, synthetic construct
  source 1..26: mol_type = other DNA
  modified_base 26: mod_base = OTHER; note = 2',3'-Dideoxycytidine
  modified_base 7: mod_base = OTHER; note = uracil
gagtcatagtcatctgacagaacgac

SEQ ID NO: 2 - DNA, 28 bp, synthetic construct
  source 1..28: mol_type = other DNA
  modified_base 9: mod_base = OTHER; note = n is 6-vinyl purine
  modified_base 1: mod_base = OTHER; note = 5'-amino-Peg3-deoxy-guanosine
gtcgttctntcagatgactatgactccc

SEQ ID NO: 3 - DNA, 11 bp, synthetic construct
  source 1..11: mol_type = other DNA
  modified_base 1: mod_base = OTHER; note = 5'-Phos-adenosine
aaatcgatgtg

SEQ ID NO: 4 - DNA, 11 bp, synthetic construct
  source 1..11: mol_type = other DNA
  modified_base 1: mod_base = OTHER; note = 5'-Phos-Cytidine
catcgatttgg

SEQ ID NO: 5 - DNA, 26 bp, synthetic construct
  source 1..26: mol_type = other DNA
  modified_base 26: mod_base = OTHER; note = 2',3'-Dideoxycytidine
  modified_base 1: mod_base = OTHER; note = 5'-Phos-Guanine
  modified_base 15: mod_base = OTHER; note = uracil
gagtcatagtcatctgacagaacgac

SEQ ID NO: 6 - DNA, 28 bp, synthetic construct
  source 1..28: mol_type = other DNA
  modified_base 1: mod_base = OTHER; note = 5'-(amino-Peg3)-deoxy-guanidine
  modified_base 9: mod_base = OTHER; note = n is 6-vinyl purine
gtcgttctntcagatgactatgactctg

SEQ ID NO: 7 - DNA, 29 bp, synthetic construct
  source 1..29: mol_type = other DNA
  modified_base 1: mod_base = OTHER; note = 5'-Phos-Guanine
  modified_base 29: mod_base = OTHER; note = 2',3'-Dideoxycytidine
  modified_base 15: mod_base = OTHER; note = uracil
  modified_base 20: mod_base = OTHER; note = uracil
gagtcatagtcatctgacatcgaaacgac

SEQ ID NO: 8 - DNA, 30 bp, synthetic construct
  source 1..30: mol_type = other DNA
  modified_base 1: mod_base = OTHER; note = 5'-(amino-Peg3)-deoxy-guanidine
  modified_base 11: mod_base = OTHER; note = n is 6-vinyl purine
gtcgttcgatntcagatgactatgactctg

SEQ ID NO: 9 - DNA, 52 bp, synthetic construct
  source 1..52: mol_type = other DNA
tcgtcggcagcgtcagatgtgtataagagacagtcagatgactatgactctg

SEQ ID NO: 10 - DNA, 12 bp, synthetic construct
  source 1..12: mol_type = other DNA
  modified_base 7: mod_base = OTHER; note = Biotin
tcatagtcatct